(19) United States
(12) Patent Application Publication
(10) Pub. No.: US 2016/0332976 A1
(43) Pub. Date: Nov. 17, 2016

(54) PROCESSES FOR PREPARING FUSED HETEROCYCLIC ION CHANNEL MODULATORS

(71) Applicant: Gilead Sciences, Inc., Foster City, CA (US)

(72) Inventors: Anna Chiu, Burlingame, CA (US); Yanshu Feng, Dalian (CN); Hanrong Gao, Dalian (CN); James A. Kerschen, Somerset, NJ (US); John Reichwein, Basking Ridge, NJ (US); Keshab Sarma, Sunnyvale, CA (US); Andrew S. Thompson, Mountainside, NJ (US); Xinjun Zhao, Dalian (CN)

(21) Appl. No.: 15/144,063

(22) Filed: May 2, 2016

Related U.S. Application Data
(62) Division of application No. 14/621,887, filed on Feb. 13, 2015, now abandoned.

(30) Foreign Application Priority Data
Feb. 13, 2014 (CN) 201410050699.2

(51) Int. Cl. C07D 267/14; C07D 413/06

(52) U.S. Cl. CPC C07D 267/14; C07D 413/06

(57) ABSTRACT

The present disclosure provides processes for the preparation of a compound of formula:

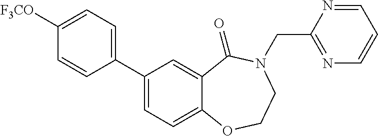 [SMILES: O=C1c2cc(-c3ccc(OC(F)(F)F)cc3)ccc2OCCN1Cc1ncccn1]

which is a selective late sodium current inhibitor. The disclosure also provides compounds that are synthetic intermediates.

### CROSS-REFERENCE TO RELATED APPLICATIONS

This application claims the benefit of Chinese priority Application Number 201410050699.2, filed on Feb. 13, 2014.

### FIELD

The present disclosure relates generally to the field of organic synthetic methodology for the preparation of a fused heterocyclic selective late sodium current inhibitor and the synthetic intermediates prepared thereby.

### BACKGROUND

The late sodium current (INaL) is a sustained component of the fast Na⁺ current of cardiac myocytes and neurons. Many common neurological and cardiac conditions are associated with abnormal (INaL) enhancement, which contributes to the pathogenesis of both electrical and contactile dysfunction in mammals. See, for example, Pathophysiology and Pharmacology of the Cardiac “Late Sodium Current”, Pharmacology and Therapeutics 119 (2008) 326-339. Accordingly, compounds that selectively inhibit (INaL) in mammals may therefore be useful in treating such disease states.

### SUMMARY

The compound of Formula XIIA is known to be a selective late sodium current inhibitor (WO 2013/006485). Processes suitable for its production are disclosed herein.

The present disclosure provides, in one embodiment, a process for making a compound of Formula (XIIA):

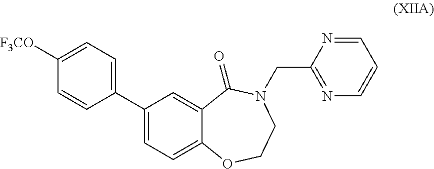 [SMILES: O=C1c2cc(-c3ccc(OC(F)(F)F)cc3)ccc2OCCN1Cc1ncccn1]

or a salt or solvate thereof.

The processes disclosed herein utilize a compound of Formula (I), or salt thereof

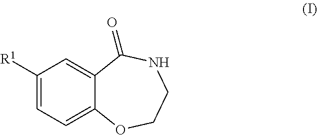

Thus, in one embodiment, provided is a process for preparing a compound of Formula (XIIA), or a salt thereof:

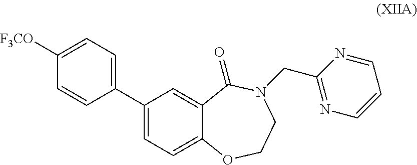 [SMILES: O=C1c2cc(-c3ccc(OC(F)(F)F)cc3)ccc2OCCN1Cc1ncccn1]

comprising the steps of:

a) contacting a compound of Formula (I), or a salt thereof:

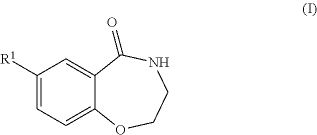

with a compound of the formula

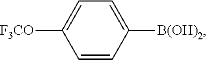 [SMILES: OB(O)c1ccc(OC(F)(F)F)cc1]

or a boronic ester thereof, under reaction conditions sufficient to provide a compound of Formula (IC), or a salt thereof and

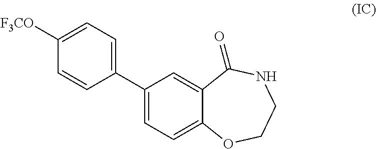 [SMILES: O=C1NCCOc2ccc(-c3ccc(OC(F)(F)F)cc3)cc21]

b) contacting the compound of Formula (IC), or a salt thereof, with a compound of the formula

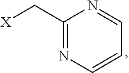

where X is halo or —S(O)₂R⁵, under reaction conditions sufficient to provide the compound of Formula (XIIA) or a salt thereof,

wherein:

R¹ is hydrogen or halo; and R⁵ is selected from the group consisting of alkyl, cycloalkyl, heterocyclyl, aryl, and heteroaryl, wherein each cycloalkyl, heterocyclyl, aryl, and heteroaryl is optionally substituted with one to three C₁₋₄ alkyl.

In another embodiment, provided is a process for preparing a compound of Formula (XII) or a salt thereof:

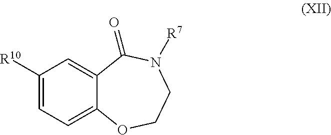

comprising the steps of:

a) cyclizing a compound of Formula (III) or a salt thereof, under reaction conditions sufficient to provide the compound of Formula (I) or a salt thereof:

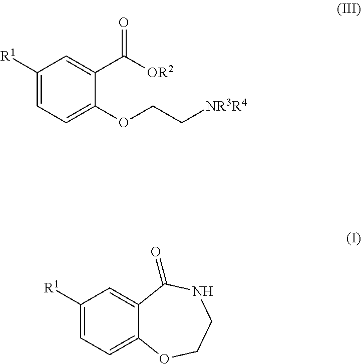

b) contacting the compound of Formula (I), or a salt thereof, with a compound of the formula X—R⁷, where X is halo or —S(O)₂R⁵, under reaction conditions sufficient to provide the compound of Formula (XII) or a salt thereof, wherein:

R¹ is hydrogen or halo;

R² is hydrogen or alkyl optionally substituted with aryl;

R³ is hydrogen or a nitrogen protecting group;

R⁴ is hydrogen, or R³ and R⁴ together with the nitrogen to which they are attached form N-diphenylmethyleneamine or a succinimide;

R⁵ is selected from the group consisting of alkyl, cycloalkyl, heterocyclyl, aryl, and heteroaryl, wherein each cycloalkyl, heterocyclyl, aryl, and heteroaryl is optionally substituted with one to three C₁₋₄ alkyl;

R⁷ is —C₁₋₆ alkylene-R⁸, -L-R⁸, alkylene-R⁸, —C₁₋₆ alkylene-L-R⁸ or alkylene-L-C₁₋₆ alkylene-R⁸;

L is —O—, —S—, —C(O)—, —NHS(O)₂—, —S(O)₂NH—, —C(O)NH— or —NHC(O)—, provided that when R⁷ is -L-R⁸ or -L-C₁₋₆ alkylene-R⁸, then L is not —O—, —S—, —NHS(O)₂— or —NHC(O)—;

R⁸ is cycloalkyl, aryl, heteroaryl or heterocyclyl; wherein said cycloalkyl, aryl, heteroaryl or heterocyclyl are optionally substituted with one, two or three substituents independently selected from the group consisting of C₁₋₆ alkyl, C₂₋₄ alkynyl, halo, —NO₂, cycloalkyl, aryl, heterocyclyl, heteroaryl, —N(R²⁰)(R²²), —N(R²⁰)—S(O)₂—R²⁰, —N(R²⁰)—C(O)—R², —C(O)—R²⁰, —C(O)—OR²⁰, —C(O)—N(R²⁰)(R²²), —CN, oxo and —O—R²⁰; wherein said C₁₋₆ alkyl, cycloalkyl, aryl, heterocyclyl or heteroaryl are optionally further substituted with one, two or three substituents independently selected from the group consisting of halo, —NO₂, C₁₋₆ alkyl, cycloalkyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, cycloalkyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;

R¹⁰ is hydrogen, halo, aryl, cycloalkyl, cycloalkenyl, heterocyclyl, or heteroaryl, wherein each aryl, cycloalkyl, cycloalkenyl, heterocyclyl, or heteroaryl is optionally substituted with one to three R¹¹;

each R¹¹ is independently halo, hydroxyl, —NO₂, —CN, —CF₃, —OCF₃, —Si(CH₃)₃, C₁₋₄ alkyl, C₁₋₃ alkoxy, C₂₋₄ alkenyl, C₂₋₄ alkynyl, aralkyl, aryloxy, aralkyloxy, acyl, carboxy, carboxyester, acylamino, amino, substituted amino, cycloalkyl, aryl, heteroaryl and heterocyclyl;

when R²⁰ and R²² are attached to a common nitrogen atom R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₃ alkoxy, —CF₃, —OCF₃, aryl, heteroaryl and cycloalkyl; and

each R²⁶ is independently selected from the group consisting of hydrogen, C₁₋₄ alkyl, aryl and cycloalkyl; wherein the C₁₋₄ alkyl, aryl and cycloalkyl may be further substituted with from 1 to 3 substituents independently selected from the group consisting of hydroxyl, halo, C₁₋₄ alkoxy, —CF₃ and —OCF₃.

Also provided are processes for preparing a compound of Formula (I), or salt thereof. In one embodiment, provided is a process for preparing a compound of Formula (I) or a salt thereof:

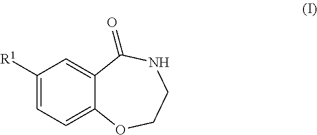

comprising cyclizing a compound of Formula (III) or a salt thereof:

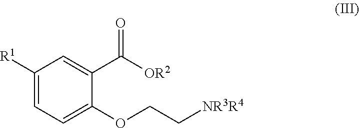

under reaction conditions sufficient to provide the compound of Formula (I) or a salt thereof, wherein:

R¹ is hydrogen or halo;

R² is hydrogen or alkyl optionally substituted with aryl;

R³ is hydrogen or a nitrogen protecting group; and

R⁴ is hydrogen, or R³ and R⁴ together with the nitrogen to which they are attached form N-diphenylmethyleneamine or a succinimide.

In another embodiment, provided is a process for preparing a compound of Formula (I) or a salt thereof:

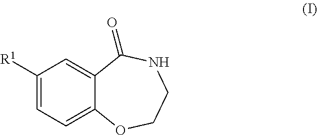

comprising:

a) deprotecting a compound of Formula (III) or a salt thereof:

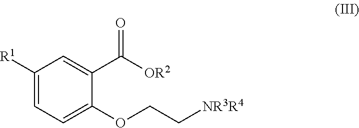

under reaction conditions sufficient to provide a compound of Formula (II) or a salt thereof, and

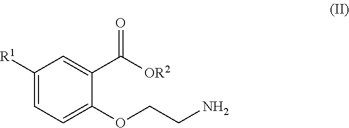

b) cyclizing a compound of formula (II) or a salt thereof, under reaction conditions sufficient to provide the compound of Formula (I) or a salt thereof, wherein:

R¹ is hydrogen or halo;

R² is hydrogen or alkyl optionally substituted with aryl;

R³ is a nitrogen protecting group; and

R⁴ is hydrogen, or R³ and R⁴ together with the nitrogen to which they are attached form N-diphenylmethyleneamine or a succinimide.

In yet another embodiment, provided is a process for preparing a compound of Formula (I) or a salt thereof:

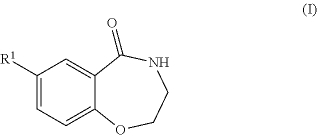

comprising contacting a compound of Formula (VI) or a salt thereof:

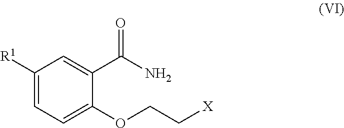

with a base, under reaction conditions sufficient to provide the compound of Formula (I) or a salt thereof, wherein:

R¹ is hydrogen or halo;

X is halo or —S(O)₂R⁵; and

R⁵ is selected from the group consisting of alkyl, cycloalkyl, heterocyclyl, aryl, and heteroaryl, wherein each cycloalkyl, heterocyclyl, aryl, and heteroaryl is optionally substituted with one to three C₁₋₄ alkyl.

In yet another embodiment, provided is a process for preparing a compound of Formula (I) or a salt thereof:

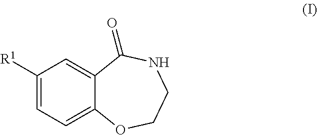

comprising contacting a compound of Formula (VIII) or a salt thereof:

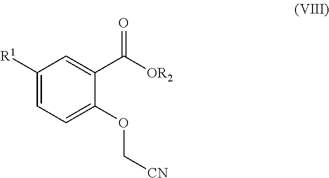

with a reducing agent, under reaction conditions sufficient to provide the compound of Formula (II) or a salt thereof,

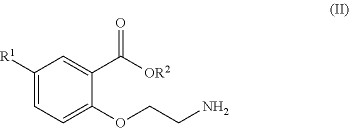

and cyclizing a compound of Formula (II) or a salt thereof to provide the compound of formula (I) or a salt thereof, wherein:

R¹ is hydrogen or halo; and

R² is hydrogen or alkyl optionally substituted with aryl.

In another embodiment, provided is a process for preparing a compound of Formula (I) or a salt thereof:

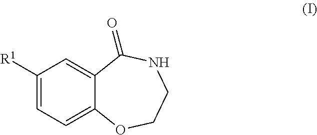

comprising contacting a compound of Formula (IX) or a salt thereof:

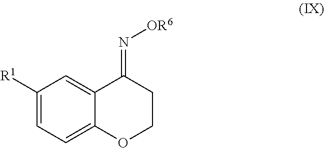

with an acid under reaction conditions sufficient to provide a compound of Formula (I) or a salt thereof, wherein:

R¹ is hydrogen or halo;

R⁶ is hydrogen or —S(O)₂R⁵; and

R⁵ is selected from the group consisting of alkyl, cycloalkyl, heterocyclyl, aryl, and heteroaryl, wherein each cycloalkyl, heterocyclyl, aryl, and heteroaryl is optionally substituted with one to three C₁₋₄ alkyl.

In another embodiment, provided is a process for preparing a compound of Formula (I) or a salt thereof:

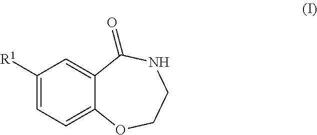

or a salt thereof, comprising contacting a compound of Formula (XI) or a salt thereof:

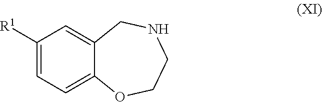

with an oxidant under reaction conditions sufficient to provide the compound of Formula (I) or a salt thereof, wherein:

R¹ is hydrogen or halo; and

R² is hydrogen or alkyl optionally substituted with aryl.

In another embodiment, provided is a process for preparing a compound of Formula (IA), or a salt thereof:

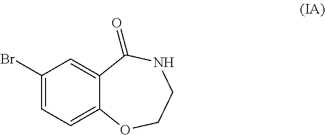 [SMILES: O=C1NCCOc2ccc(Br)cc21]

comprising contacting a compound of Formula (IB), or a salt thereof:

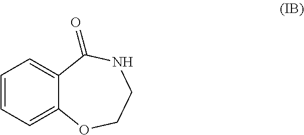 [SMILES: O=C1NCCOc2ccccc21]

with Br₂, under reaction conditions sufficient to provide a compound of Formula (IA), or a salt thereof.

In other embodiments, the disclosure provides intermediate compounds that may be used in the processes described herein. Thus, for instance, one embodiment is a compound of the formula:

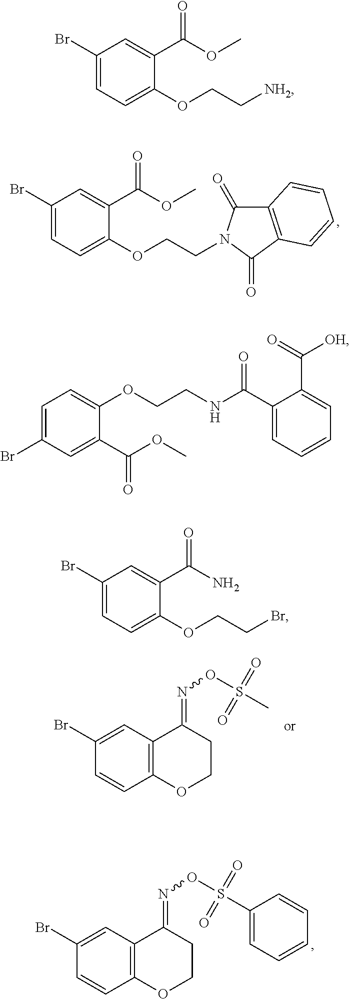

or a salt thereof.

The inventions of this disclosure are described throughout. In addition, specific embodiments of the invention are as disclosed herein.

### DETAILED DESCRIPTION

### 1. Definitions and General Parameters

As used in the present specification, the following words and phrases are generally intended to have the meanings as set forth below, except to the extent that the context in which they are used indicates otherwise.

The term “alkyl” refers to a monoradical branched or unbranched saturated hydrocarbon chain having from 1 to 20 carbon atoms, or from 1 to 15 carbon atoms, or from 1 to 10 carbon atoms, or from 1 to 8 carbon atoms, or from 1 to 6 carbon atoms, or from 1 to 4 carbon atoms.

This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:
1) an alkyl group as defined above, having 1, 2, 3, 4 or 5 substituents, (in some embodiments, 1, 2 or 3 substituents) selected from the group consisting of alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2; or
2) an alkyl group as defined above that is interrupted by 1-10 atoms (e.g. 1, 2, 3, 4 or 5 atoms) independently chosen from oxygen, sulfur and NR^{a}, where R^{a} is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)R^{a}, in which R^{a} is alkyl, aryl or heteroaryl and n is 0, 1 or 2; or
3) an alkyl group as defined above that has both 1, 2, 3, 4 or 5 substituents as defined above and is also interrupted by 1-10 atoms (e.g. 1, 2, 3, 4 or 5 atoms) as defined above.

The term “lower alkyl” refers to a monoradical branched or unbranched saturated hydrocarbon chain having 1, 2, 3, 4, 5 or 6 carbon atoms. This term is exemplified by groups such as methyl, ethyl, n-propyl, iso-propyl, n-butyl, iso-butyl, t-butyl, n-hexyl, and the like.

The term “substituted lower alkyl” refers to lower alkyl as defined above having 1 to 5 substituents (in some embodiments, 1, 2 or 3 substituents), as defined for substituted alkyl or a lower alkyl group as defined above that is interrupted by 1, 2, 3, 4 or 5 atoms as defined for substituted alkyl or a lower alkyl group as defined above that has both 1, 2, 3, 4 or 5 substituents as defined above and is also interrupted by 1, 2, 3, 4 or 5 atoms as defined above.

The term “alkylene” refers to a diradical of a branched or unbranched saturated hydrocarbon chain, in some embodiments, having from 1 to 20 carbon atoms (e.g. 1-10 carbon atoms or 1, 2, 3, 4, 5 or 6 carbon atoms). This term is exemplified by groups such as methylene (—CH₂—), ethylene (—CH₂CH₂—), the propylene isomers (e.g., —CH₂CH₂CH₂— and —CH(CH₃)CH₂—), and the like.

The term “lower alkylene” refers to a diradical of a branched or unbranched saturated hydrocarbon chain, in some embodiments, having 1, 2, 3, 4, 5 or 6 carbon atoms.

The term “substituted alkylene” refers to an alkylene group as defined above having 1 to 5 substituents (in some embodiments, 1, 2 or 3 substituents) as defined for substituted alkyl.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “aralkyloxy” refers to the group —O-aralkyl. “Optionally substituted aralkyloxy” refers to an optionally substituted aralkyl group covalently linked to an optionally substituted alkylene group. Such aralkyl groups are exemplified by benzyloxy, phenylethyloxy, and the like.

The term “alkenyl” refers to a monoradical of a branched or unbranched unsaturated hydrocarbon group having from 2 to 20 carbon atoms (in some embodiments, from 2 to 10 carbon atoms, e.g. 2 to 6 carbon atoms) and having from 1 to 6 carbon-carbon double bonds, e.g. 1, 2 or 3 carbon-carbon double bonds. In some embodiments, alkenyl groups include ethenyl (or vinyl, i.e. —CH═CH₂), 1-propylene (or allyl, i.e. —CH₂CH═CH₂), isopropylene (—C(CH₃)═CH₂), and the like.

The term “lower alkenyl” refers to alkenyl as defined above having from 2 to 6 carbon atoms.

The term “substituted alkenyl” refers to an alkenyl group as defined above having 1 to 5 substituents (in some embodiments, 1, 2 or 3 substituents) as defined for substituted alkyl.

The term “alkenylene” refers to a diradical of a branched or unbranched unsaturated hydrocarbon group having from 2 to 20 carbon atoms (in some embodiments, from 2 to 10 carbon atoms, e.g. 2 to 6 carbon atoms) and having from 1 to 6 carbon-carbon double bonds, e.g. 1, 2 or 3 carbon-carbon double bonds.

The term “alkynyl” refers to a monoradical of an unsaturated hydrocarbon, in some embodiments, having from 2 to 20 carbon atoms (in some embodiments, from 2 to 10 carbon atoms, e.g. 2 to 6 carbon atoms) and having from 1 to 6 carbon-carbon triple bonds e.g. 1, 2 or 3 carbon-carbon triple bonds. In some embodiments, alkynyl groups include ethynyl (—C≡CH), propargyl (or propynyl, i.e. —C≡C≡CH₃), and the like.

The term “substituted alkynyl” refers to an alkynyl group as defined above having 1 to 5 substituents (in some embodiments, 1, 2 or 3 substituents) as defined for substituted alkyl.

The term “alkynylene” refers to a diradical of an unsaturated hydrocarbon, in some embodiments, having from 2 to 20 carbon atoms (in some embodiments, from 2 to 10 carbon atoms, e.g. 2 to 6 carbon atoms) and having from 1 to 6 carbon-carbon triple bonds e.g. 1, 2 or 3 carbon-carbon triple bonds.

The term “benzyl” refers to the group —CH₂—C₆Hs.

The term “hydroxy” or “hydroxyl” refers to a group —OH.

The term “alkoxy” refers to the group R—O—, where R is alkyl or —Y—Z, in which Y is alkylene and Z is alkenyl or alkynyl, where alkyl, alkenyl and alkynyl are as defined herein. In some embodiments, alkoxy groups are alkyl-O— and includes, by way of example, methoxy, ethoxy, n-propoxy, iso-propoxy, n-butoxy, tert-butoxy, sec-butoxy, n-pentoxy, n-hexyloxy, 1,2-dimethylbutoxy, and the like.

The term “lower alkoxy” refers to the group R—O— in which R is optionally substituted lower alkyl. This term is exemplified by groups such as methoxy, ethoxy, n-propoxy, iso-propoxy, n-butoxy, iso-butoxy, t-butoxy, n-hexyloxy, and the like.

The term “substituted alkoxy” refers to the group R—O—, where R is substituted alkyl or —Y—Z, in which Y is substituted alkylene and Z is substituted alkenyl or substituted alkynyl, where substituted alkyl, substituted alkenyl and substituted alkynyl are as defined herein.

The term “C₁₋₃ haloalkyl” refers to an alkyl group having from 1 to 3 carbon atoms covalently bonded to from 1 to 7, or from 1 to 6, or from 1 to 3, halogen(s), where alkyl and halogen are defined herein. In some embodiments, C₁₋₃ haloalkyl includes, by way of example, trifluoromethyl, difluoromethyl, fluoromethyl, 2,2,2-trifluoroethyl, 2,2-difluoroethyl, 2-fluoroethyl, 3,3,3-trifluoropropyl, 3,3-difluoropropyl, 3-fluoropropyl.

The term “cycloalkyl” refers to cyclic alkyl groups of from 3 to 20 carbon atoms, or from 3 to 10 carbon atoms, having a single cyclic ring or multiple condensed rings. Such cycloalkyl groups include, by way of example, single ring structures such as cyclopropyl, cyclobutyl, cyclopentyl, cyclooctyl and the like or multiple ring structures such as adamantanyl and bicyclo[2.2.1]heptanyl or cyclic alkyl groups to which is fused an aryl group, for example indanyl, and the like, provided that the point of attachment is through the cyclic alkyl group.

The term “cycloalkenyl” refers to cyclic alkyl groups of from 3 to 20 carbon atoms having a single cyclic ring or multiple condensed rings and having at least one double bond and in some embodiments, from 1 to 2 double bonds.

The terms “substituted cycloalkyl” and “susbstituted cycloalkenyl” refer to cycloalkyl or cycloalkenyl groups having 1, 2, 3, 4 or 5 substituents (in some embodiments, 1, 2 or 3 substituents), selected from the group consisting of alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)— heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. The term “substituted cycloalkyl” also includes cycloalkyl groups wherein one or more of the annular carbon atoms of the cycloalkyl group has an oxo group bonded thereto. In addition, a substituent on the cycloalkyl or cycloalkenyl may be attached to the same carbon atom as, or is geminal to, the attachment of the substituted cycloalkyl or cycloalkenyl to the 6,7-ring system. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “cycloalkoxy” refers to the group cycloalkyl-O—.

The term “substituted cycloalkoxy” refers to the group substituted cycloalkyl-O—.

The term “cycloalkenyloxy” refers to the group cycloalkenyl-O—.

The term “substituted cycloalkenyloxy” refers to the group substituted cycloalkenyl-O—.

The term “aryl” refers to an aromatic carbocyclic group of 6 to 20 carbon atoms having a single ring (e.g., phenyl) or multiple rings (e.g., biphenyl) or multiple condensed (fused) rings (e.g., naphthyl, fluorenyl and anthryl). In some embodiments, aryls include phenyl, fluorenyl, naphthyl, anthryl, and the like.

Unless otherwise constrained by the definition for the aryl substituent, such aryl groups may optionally be substituted with 1, 2, 3, 4 or 5 substituents (in some embodiments, 1, 2 or 3 substituents), selected from the group consisting of alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)— heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “aryloxy” refers to the group aryl-O— wherein the aryl group is as defined above, and includes optionally substituted aryl groups as also defined above. The term “arylthio” refers to the group R—S—, where R is as defined for aryl.

The term “heterocyclyl,” “heterocycle,” or “heterocyclic” refers to a monoradical saturated group having a single ring or multiple condensed rings, having from 1 to 40 carbon atoms and from 1 to 10 hetero atoms, and from 1 to 4 heteroatoms, selected from nitrogen, sulfur, phosphorus, and/or oxygen within the ring. In some embodiments, the “heterocyclyl,” “heterocycle,” or “heterocyclic” group is linked to the remainder of the molecule through one of the heteroatoms within the ring.

Unless otherwise constrained by the definition for the heterocyclic substituent, such heterocyclic groups may be optionally substituted with 1 to 5 substituents (in some embodiments, 1, 2 or 3 substituents), selected from the group consisting of alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. In addition, a substituent on the heterocyclic group may be attached to the same carbon atom as, or is geminal to, the attachment of the substituted heterocyclic group to the 6,7-ring system. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2. Examples of heterocyclics include tetrahydrofuranyl, morpholino, piperidinyl, and the like.

The term “heterocyclooxy” refers to the group —O-heterocyclyl.

The term “heteroaryl” refers to a group comprising single or multiple rings comprising 1 to 15 carbon atoms and 1 to 4 heteroatoms selected from oxygen, nitrogen and sulfur within at least one ring. The term “heteroaryl” is generic to the terms “aromatic heteroaryl” and “partially saturated heteroaryl”. The term “aromatic heteroaryl” refers to a heteroaryl in which at least one ring is aromatic, regardless of the point of attachment. Examples of aromatic heteroaryls include pyrrole, thiophene, pyridine, quinoline, pteridine.

The term “partially saturated heteroaryl” refers to a heteroaryl having a structure equivalent to an underlying aromatic heteroaryl which has had one or more double bonds in an aromatic ring of the underlying aromatic heteroaryl saturated. Examples of partially saturated heteroaryls include dihydropyrrole, dihydropyridine, chroman, 2-oxo-1,2-dihydropyridin-4-yl, and the like.

Unless otherwise constrained by the definition for the heteroaryl substituent, such heteroaryl groups may be optionally substituted with 1 to 5 substituents (in some embodiments, 1, 2 or 3 substituents) selected from the group consisting alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)— heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2. Such heteroaryl groups can have a single ring (e.g., pyridyl or furyl) or multiple condensed rings (e.g., indolizinyl, benzothiazole or benzothienyl). Examples of nitrogen heterocyclyls and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, and the like as well as N-alkoxy-nitrogen containing heteroaryl compounds.

The term “heteroaryloxy” refers to the group heteroaryl-O—.

The term “amino” refers to the group —NH₂.

The term “substituted amino” refers to the group —NRR where each R is independently selected from the group consisting of hydrogen, alkyl, cycloalkyl, aryl, heteroaryl and heterocyclyl provided that both R groups are not hydrogen or a group —Y—Z, in which Y is optionally substituted alkylene and Z is alkenyl, cycloalkenyl or alkynyl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “alkyl amine” refers to R—NH₂ in which R is optionally substituted alkyl.

The term “dialkyl amine” refers to R—NHR in which each R is independently an optionally substituted alkyl.

The term “trialkyl amine” refers to NR₃ in which each R is independently an optionally substituted alkyl.

The term “cyano” refers to the group —CN.

The term “azido” refers to a group

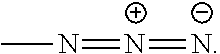

The term “keto” or “oxo” refers to a group ═O.

The term “carboxy” refers to a group —C(O)—OH.

The term “ester” or “carboxyester” refers to the group —C(O)OR, where R is alkyl, cycloalkyl, aryl, heteroaryl or heterocyclyl, which may be optionally further substituted by alkyl, alkoxy, halogen, CF₃, amino, substituted amino, cyano or —S(O)R^{a}, in which R^{a} is alkyl, aryl or heteroaryl and n is 0, 1 or 2.

The term “acyl” denotes the group —C(O)R, in which R is hydrogen, alkyl, cycloalkyl, heterocyclyl, aryl or heteroaryl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)R^{a}, in which R^{a} is alkyl, aryl or heteroaryl and n is 0, 1 or 2.

The term “carboxyalkyl” refers to the groups —C(O)O-alkyl or —C(O)O-cycloalkyl, where alkyl and cycloalkyl are as defined herein, and may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “aminocarbonyl” refers to the group —C(O)NRR where each R is independently hydrogen, alkyl, cycloalkyl, aryl, heteroaryl, or heterocyclyl, or where both R groups are joined to form a heterocyclic group (e.g., morpholino). Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “acyloxy” refers to the group —OC(O)—R, in which R is alkyl, cycloalkyl, heterocyclyl, aryl or heteroaryl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “acylamino” refers to the group —NRC(O)R where each R is independently hydrogen, alkyl, cycloalkyl, aryl, heteroaryl or heterocyclyl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “alkoxycarbonylamino” refers to the group —N(R^{d})C(O)OR in which R is alkyl and R^{d} is hydrogen or alkyl. Unless otherwise constrained by the definition, each alkyl may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)R^{a}, in which R^{a} is alkyl, aryl or heteroaryl and n is 0, 1 or 2.

The term “aminocarbonylamino” refers to the group —NR^{c}C(O)NRR, wherein R^{c} is hydrogen or alkyl and each R is hydrogen, alkyl, cycloalkyl, aryl, heteroaryl or heterocyclyl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)R^{a}, in which R^{a} is alkyl, aryl or heteroaryl and n is 0, 1 or 2.

The term “thiol” refers to the group —SH.

The term “thiocarbonyl” refers to a group ═S.

The term “alkylthio” refers to the group —S-alkyl.

The term “substituted alkylthio” refers to the group —S-substituted alkyl.

The term “heterocyclylthio” refers to the group —S-heterocyclyl.

The term “arylthio” refers to the group —S-aryl.

The term “heteroarylthiol” refers to the group —S-heteroaryl wherein the heteroaryl group is as defined above including optionally substituted heteroaryl groups as also defined above.

The term “sulfoxide” refers to a group —S(O)R, in which R is alkyl, cycloalkyl, heterocyclyl, aryl or heteroaryl. “Substituted sulfoxide” refers to a group —S(O)R, in which R is substituted alkyl, substituted cycloalkyl, substituted heterocyclyl, substituted aryl or substituted heteroaryl, as defined herein.

The term “sulfone” refers to a group —S(O)₂R, in which R is alkyl, cycloalkyl, heterocyclyl, aryl or heteroaryl. “Substituted sulfone” refers to a group —S(O)₂R, in which R is substituted alkyl, substituted cycloalkyl, substituted heterocyclyl, substituted aryl or substituted heteroaryl, as defined herein.

The term “aminosulfonyl” refers to the group —S(O)₂NRR, wherein each R is independently hydrogen, alkyl, cycloalkyl, aryl, heteroaryl or heterocyclyl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “hydroxyamino” refers to the group —NHOH.

The term “alkoxyamino” refers to the group —NHOR in which R is optionally substituted alkyl.

The term “halogen” or “halo” refers to fluoro, bromo, chloro and iodo.

“Optional” or “optionally” means that the subsequently described event or circumstance may or may not occur, and that the description includes instances where said event or circumstance occurs and instances in which it does not.

A “substituted” group includes embodiments in which a monoradical substituent is bound to a single atom of the substituted group (e.g. forming a branch), and also includes embodiments in which the substituent may be a diradical bridging group bound to two adjacent atoms of the substituted group, thereby forming a fused ring on the substituted group.

Where a given group (moiety) is described herein as being attached to a second group and the site of attachment is not explicit, the given group may be attached at any available site of the given group to any available site of the second group. For example, a “lower alkyl-substituted phenyl”, where the attachment sites are not explicit, may have any available site of the lower alkyl group attached to any available site of the phenyl group. In this regard, an “available site” is a site of the group at which a hydrogen of the group may be replaced with a substituent.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. Also not included are infinite numbers of substituents, whether the substituents are the same or different. In such cases, the maximum number of such substituents is three. Each of the above definitions is thus constrained by a limitation that, for example, substituted aryl groups are limited to -substituted aryl-(substituted aryl)-substituted aryl.

A compound of a given formula is intended to encompass the compounds of the disclosure, and the pharmaceutically acceptable salts, pharmaceutically acceptable esters, isomers, tautomers, solvates, isotopes, hydrates, polymorphs, and prodrugs of such compounds, unless the context suggests otherwise. Additionally, the compounds of the disclosure may possess one or more asymmetric centers, and may be produced as a racemic mixture or as individual enantiomers or diastereoisomers. The number of stereoisomers present in any given compound of a given formula depends upon the number of asymmetric centers present (there are 2ⁿ stereoisomers possible where n is the number of asymmetric centers). The individual stereoisomers may be obtained by resolving a racemic or non-racemic mixture of an intermediate at some appropriate stage of the synthesis or by resolution of the compound by conventional means. The individual stereoisomers (including individual enantiomers and diastereoisomers) as well as racemic and non-racemic mixtures of stereoisomers are encompassed within the scope of the present disclosure, all of which are intended to be depicted by the structures of this specification unless otherwise specifically indicated.

“Isomers” are different compounds that have the same molecular formula. Isomers include stereoisomers, enantiomers and diastereomers.

“Stereoisomers” are isomers that differ only in the way the atoms are arranged in space.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate.

“Diastereoisomers” are stereoisomers that have at least two asymmetric atoms, but which are not mirror-images of each other.

The absolute stereochemistry is specified according to the Cahn Ingold Prelog R S system. When the compound is a pure enantiomer the stereochemistry at each chiral carbon may be specified by either R or S. Resolved compounds whose absolute configuration is unknown are designated (+) or (−) depending on the direction (dextro- or laevorotary) that they rotate the plane of polarized light at the wavelength of the sodium D line.

Some of the compounds exist as tautomeric isomers. Tautomeric isomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers. Non-limiting examples of amide-comprising and imidic acid-comprising tautomers are shown below:

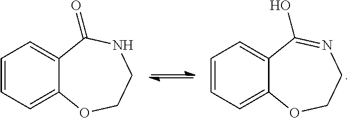 [SMILES: O=C1NCCOc2ccccc21 ; OC1=NCCOc2ccccc21]

The term “polymorph” refers to different crystal structures of a crystalline compound. The different polymorphs may result from differences in crystal packing (packing polymorphism) or differences in packing between different conformers of the same molecule (conformational polymorphism).

The term “solvate” refers to a complex formed by the combining of a compound and a solvent.

The term “hydrate” refers to the complex formed by the combining of a compound and water.

The term “prodrug” refers to compounds that include chemical groups which, in vivo, can be converted and/or can be split off from the remainder of the molecule to provide for the active drug, a pharmaceutically acceptable salt thereof or a biologically active metabolite thereof.

Any formula or structure given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that may be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure may include, for example, those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes compounds in which from 1 to n hydrogens attached to a carbon atom is/are replaced by deuterium, in which n is the number of hydrogens in the molecule. Such compounds may exhibit increased resistance to metabolism and may thus be useful for increasing the half life of a compound intended for use in a mammal. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies.

Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base “salts” by virtue of the presence of amino and/or carboxyl groups or groups similar thereto. In some cases, the “salt” of a given compound is a pharmaceutically acceptable salt. The term “pharmaceutically acceptable salt” of a given compound refers to salts that retain the biological effectiveness and properties of the given compound, and which are not biologically or otherwise undesirable.

Base addition salts may be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Amines are of general structure N(R³⁰)(R³¹)(R³²), wherein mono-substituted amines have 2 of the three substituents on nitrogen (R³⁰, R³¹ and R³²) as hydrogen, di-substituted amines have 1 of the three substituents on nitrogen (R³⁰, R³¹ and R³²) as hydrogen, whereas tri-substituted amines have none of the three substituents on nitrogen (R³⁰, R³¹ and R³²) as hydrogen. R³⁰, R³¹ and R³² are selected from a variety of substituents such as hydrogen, optionally substituted alkyl, aryl, heteroayl, cycloalkyl, cycloalkenyl, heterocyclyl and the like. The above-mentioned amines refer to the compounds wherein either one, two or three substituents on the nitrogen are as listed in the name. For example, the term “cycloalkenyl amine” refers to cycloalkenyl-NH₂, wherein “cycloalkenyl” is as defined herein. The term “diheteroarylamine” refers to NH(heteroaryl)₂, wherein “heteroaryl” is as defined herein and so on. Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The term “reaction conditions” is intended to refer to the physical and/or environmental conditions under which a chemical reaction proceeds. Examples of reaction conditions include, but are not limited to, one or more of following: reaction temperature, solvent, pH, pressure, reaction time, mole ratio of reactants, the presence of a base or acid, or catalyst, radiation, etc. Reaction conditions may be named after the particular chemical reaction in which the conditions are employed, such as, coupling conditions, hydrogenation conditions, acylation conditions, reduction conditions, etc. Reaction conditions for most reactions are generally known to those skilled in the art or can be readily obtained from the literature. Examplary reaction conditions sufficient for performing the chemical transformations provided herein can be found throughout, and in particular, the examples below. It is also contemplated that the reaction conditions may include reagents in addition to those listed in the specific reaction.

The term “reducing agent” refers to the addition of hydrogen to a molecule. Exemplary reducing agents include hydrogen gas (H₂) and hydride reagents such as borohydrides, lithium aluminium hydride, diisobutylaluminium hydride (DIBAL-H) and super hydride.

The term “nitrogen protecting group” refers to a chemical moiety which is added to, and later removed from, an amine functionality to obtain chemoselectivity in a subsequent chemical reaction. The term “deprotecting” refers to removing the nitrogen protecting group. Suitable nitrogen protecting groups include carbobenzyloxy (Cbz) (removed by hydrogenolysis), p-methoxybenzyl carbonyl (Moz or MeOZ) (removed by hydrogenolysis), tert-butyloxycarbonyl (Boc) (removed by concentrated strong acids, such as HCl or trifluoroacetic acid, or by heating), 9-fluorenylmethyloxycarbonyl (FMOC) (removed by base, such as piperidine), acetyl (Ac) (removed by treatment with a base), benzoyl (Bz) (removed by treatment with a base, most often with aqueous or gaseous ammonia or methylamine), benzyl (Bn) (removed by hydrogenolysis), a carbamate (removed by acid and mild heating), p-methoxybenzyl (PMB) (removed by hydrogenolysis), 3,4-dimethoxybenzyl (DMPM) (removed by hydrogenolysis), p-methoxyphenyl (PMP) (removed by ammonium cerium(IV) nitrate), a succinimide (i.e., a cyclic imide) (removed by treatment with a base), tosyl (Ts) (removed by concentrated acid and strong reducing agents), and other sulfonamides (Nosyl and Nps) (removed by samarium iodide, tributyltin hydride, etc.).

The term “succinimide” refers to a cyclic imide, and may be monocyclic, bicyclic (e.g., phthalimides) or polycyclic, and may further be optionally substituted. Non limiting examples include N-pthalimide, N-dichlorophthalimide, N-tetrachlorophthalimide, N-4-nitrophthalimide, N-dithiasuccinimide, N-2,3-diphenylmaleimide, and N-2,3-dimethylmaleimide.

The term “catalyst” refers to a chemical substance that enables a chemical reaction to proceed at a usually faster rate or under different conditions (such as at a lower temperature) than otherwise possible.

In addition, abbreviations as used herein have respective meanings as follows:

| | |
|---|---|
| 9-BBN | 9-Borabicyclo[3.3.1]nonane |
| Ac | Acetate |
| Amphos | Bis(di-tert-butyl(4- |
| | dimethylaminophenyl)phosphine)dichloropalladium(II) |
| AN | Peak area normalization |
| aq | Aqueous |
| Boc | tert-Butoxycarbonyl |
| brs | Broad singlet |
| Bu | Butyl |
| conc. | Concentrated |
| d | Doublet |
| DBU | 1,8-Diazabicyclo[5.4.0]undec-7-ene |
| DCM | Dichloromethane |
| dd | Doublet of doublets |
| DIBAL-H | Diisobutylaluminium hydride |
| DMA | Dimethylamine |
| DMAP | 4-Dimethylaminopyridine |
| DMF | Dimethylformamide |
| DMS | Dimethyl sulfide |
| DMSO | Dimethylsulfoxide |
| equiv | Equivalents |
| Et | Ethyl |
| g | Gram |
| h | Hour |
| HDMS | Hexamethyldisilazide |
| HPLC | High-pressure liquid chromatography |
| Hz | Hertz |
| iPr | Isopropyl |
| J | Coupling constant |
| LCMS | Liquid chromatography-mass spectrometry |
| m | Multiplet |
| M | Molar |
| m/z | Mass to charge |
| Me | Methyl |
| mg | Milligram |
| MHz | Mega hertz |
| mL/ml | Milliliter |
| mmole | Millimole |
| MTBE | Methyl-tert-butyl ether |
| NMP | N-Methyl-2-pyrrolidone |
| NMR | Nuclear magnetic resonance |
| PSI/psi | Pound-force per square inch |
| Py | Pyridine |
| Red-Al | Sodium bis(2- |
| | methoxyethoxy)aluminumhydride |
| s | Singlet |
| t | Triplet |
| t-Bu | tert-Butyl |
| TEMPO | (2,2,6,6-Tetramethylpiperidin-1-yl)oxy |
| TFA | Trifluoroacetic acid |
| THF | Tetrahydrofuran |
| Ts | Tosyl |
| vol | Volume |
| wt | Weight |
| δ | Chemical shift |
| μL | Microliter |

### 2. Processes

As described generally above, the disclosure provides in some embodiments processes for making a compound of Formula I. In one embodiment, the present disclosure provides for a process for preparing a compound of Formula (I) or a salt thereof:

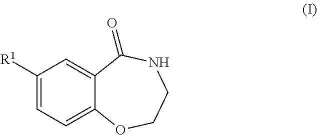

comprising cyclizing a compound of Formula (III) or a salt thereof:

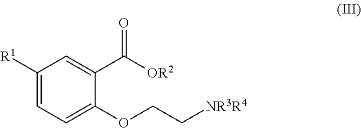

under reaction conditions sufficient to provide the compound of Formula (I) or a salt thereof, wherein:

R¹ is hydrogen or halo;

R² is hydrogen or alkyl optionally substituted with aryl;

R³ is hydrogen or a nitrogen protecting group; and

R⁴ is hydrogen, or R³ and R⁴ together with the nitrogen to which they are attached form N-diphenylmethyleneamine or a succinimide.

In one embodiment, the compound of Formula (III) is the HCl salt. In another embodiment, R¹ is bromo.

In one embodiment, the reaction conditions comprise deprotecting the compound of Formula (III) to provide a compound of Formula (II):

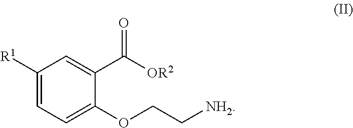

In certain embodiments, the reaction conditions comprise a base selected from the group consisting of sodium hydride, methylamine, N¹,N¹-dimethylpropane-1,3-diamine, triethylamine, diisopropylethylamine, pyridine, 1,8-diazabicyclo[5.4.0]undec-7-ene, tetrahydrofuran, 2-methyltetrahydrofuran, sodium hexamethyldisilazide, and sodium methoxide (CH₃ONa). In some embodiments, the reaction conditions comprise toluene, benzene, or xylenes, and a temperature of from about 60° C. to about 150° C., from about 95° C. to about 150° C., from about 125° C. to about 130° C., or from about 75° C. to about 85° C.

In one embodiment, provided is a process for preparing a compound of Formula (II) or a salt thereof:

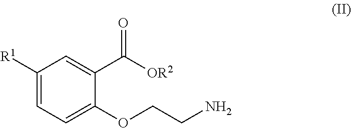

comprising deprotecting a compound of Formula (III) or a salt thereof:

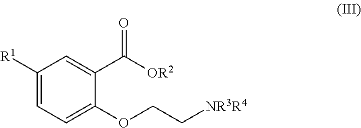

under reaction conditions sufficient to provide the compound of Formula (II) or a salt thereof, wherein:

R¹ is hydrogen or halo;

R² is hydrogen or alkyl optionally substituted with aryl;

R³ is a nitrogen protecting group; and

R⁴ is hydrogen, or R³ and R⁴ together with the nitrogen to which they are attached form N-diphenylmethyleneamine or a succinimide.

In one embodiment, R¹ is bromo. In certain embodiments, R³ and R⁴ together with the nitrogen to which they are attached form a succinimide.

In one embodiment, provided is a process for preparing a compound of Formula (I) or a salt thereof:

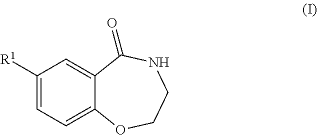

comprising:

a) deprotecting a compound of Formula (III) or a salt thereof:

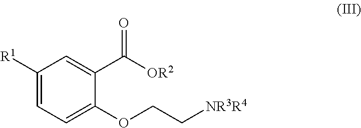

under reaction conditions sufficient to provide a compound of Formula (II) or a salt thereof, and

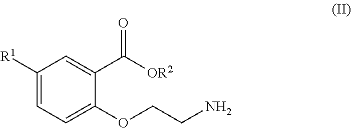

b) cyclizing a compound of formula (II) or a salt thereof, under reaction conditions sufficient to provide the compound of Formula (I) or a salt thereof, wherein:

R¹ is hydrogen or halo;

R² is hydrogen or alkyl optionally substituted with aryl;

R³ is a nitrogen protecting group; and

R⁴ is hydrogen, or R³ and R⁴ together with the nitrogen to which they are attached form N-diphenylmethyleneamine or a succinimide.

In one embodiment, R³ is acyl, allyl, —C(O)O-alkyl, or benzyl; and R⁴ is hydrogen. In another embodiment, R³ is —C(O)O-alkyl; and R⁴ is hydrogen. In yet another embodiment, R³ is acyl; and R⁴ is hydrogen.

In certain embodiments, the deprotecting step comprises an acid selected from HCl, H₃PO₄, H₂SO₄, trifluoroacetic acid, and toluenesulfonic acid, and a solvent selected from the group consisting of methanol, ethanol, isopropanol, methyl tert-butyl ether, tetrahydrofuran, and acetic acid.

In one embodiment, R¹ is bromo. In certain embodiments, R³ and R⁴ together with the nitrogen to which they are attached form a succinimide.

In certain embodiments, the reaction conditions comprise methylamine, N¹,N¹-dimethylpropane-1,3-diamine, hydroxylamine, ethylenediamine, hydrazine or a hydrazine derivative. In some embodiments, the reaction conditions of steps a) and b) comprise ethanol, methanol, isopropyl alcohol, dimethylformamide, or acetonitrile, and a temperature of from about 20° C. to about 100° C.

In one embodiment, provided is a process for preparing a compound of Formula (III) or a salt thereof:

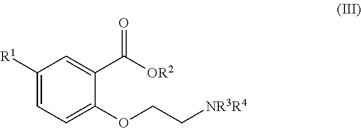

comprising coupling a compound of Formula (IV) or a salt thereof with a compound of Formula (V) or a salt thereof:

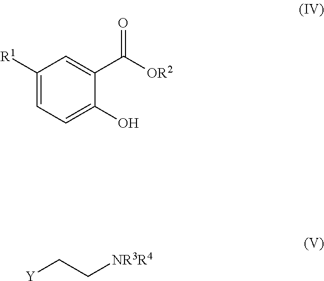

in the presence of a base, under reaction conditions sufficient to provide the compound of Formula (III) or a salt thereof;

wherein:

R¹ is hydrogen or halo;

R² is hydrogen or alkyl optionally substituted with aryl;

R³ is a nitrogen protecting group;

R⁴ is hydrogen, or R³ and R⁴ together with the nitrogen to which they are attached form N-diphenylmethyleneamine or a succinimide;

Y is halo, —OC(O)OR⁵ or —OS(O)₂R⁵; and

R⁵ is selected from the group consisting of alkyl, cycloalkyl, heterocyclyl, aryl, and heteroaryl, wherein each cycloalkyl, heterocyclyl, aryl, and heteroaryl is optionally substituted with one to three C₁₋₄ alkyl.

In one embodiment, R³ is acyl, allyl, —C(O)O-alkyl, or benzyl; and R⁴ is hydrogen. In another embodiment, R³ is —C(O)O-alkyl; and R⁴ is hydrogen. In another embodiment, R³ is acyl; and R⁴ is hydrogen. In yet another embodiment, R³ and R⁴ together with the nitrogen to which they are attached form a succinimide.

In one embodiment, the base is an organic base, an alkali metal base, a hexamethyldisilazane base, a carbonate base or an alkoxide base. In certain embodiments, the base is triethylamine, diisopropylethylamine, 1,8-diazabicyclo[5.4.0]undec-7-ene, 4-dimethylaminopyridine, sodium hydride, sodium hexamethyldisilazide, potassium hexamethyldisilazide, lithium hexamethyldisilazide, Cs₂CO₃, Na₂CO₃, or potassium tert-butoxide. In some embodiments, the reaction conditions comprise dimethylsulfoxide, dimethylformamide, dimethylacetamide, tetrahydrofuran, or N-methyl-2-pyrrolidone, and a temperature of from about 30 to about 70° C., or from about 50 to about 55° C.

In one embodiment, provided is a process for preparing a compound of Formula (I) or a salt thereof:

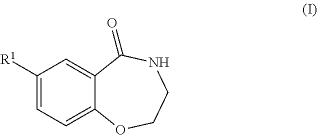

comprising contacting a compound of Formula (VI) or a salt thereof:

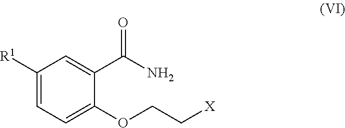

with a base, under reaction conditions sufficient to provide the compound of Formula (I) or a salt thereof, wherein:

R¹ is hydrogen or halo;

X is halo or —S(O)₂R⁵; and

R⁵ is selected from the group consisting of alkyl, cycloalkyl, heterocyclyl, aryl, and heteroaryl, wherein each cycloalkyl, heterocyclyl, aryl, and heteroaryl is optionally substituted with one to three C₁₋₄ alkyl.

In certain embodiments, the base is sodium hydride, or sodium hexamethyldisilazide. In some embodiments, the reaction conditions further comprise N,N-dimethylacetamide, dimethylformamide, N-methyl-2-pyrrolidone, or dimethylsulfoxide, and a temperature of from about −10° C. to about 40° C., or from about 20° C. to about 25° C.

In one embodiment, provided is a process for preparing a compound of Formula (VI) or a salt thereof:

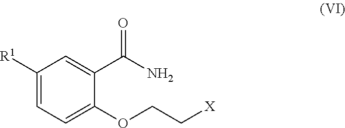

comprising contacting a compound of Formula (VII) or a salt thereof:

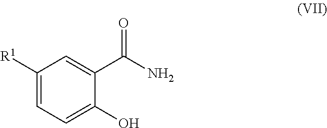

with 1,2-dibromoethane, under reaction conditions sufficient to provide the compound of Formula (VI) or a salt thereof, wherein:

R¹ is hydrogen or halo;

X is halo or —S(O)₂R⁵; and

R⁵ is selected from the group consisting of alkyl, cycloalkyl, heterocyclyl, aryl, and heteroaryl, wherein each cycloalkyl, heterocyclyl, aryl, and heteroaryl is optionally substituted with one to three C₁₋₄ alkyl.

In certain embodiments, the reaction conditions comprise a base. Suitable bases include, e.g., K₂CO₃, Na₂CO₃, Cs₂CO₃, triethylamine, sodium hydride, or sodium hexamethyldisilazide.

In certain embodiments, the reaction conditions further comprise N,N-dimethylacetamide, dimethylformamide, N-methyl-2-pyrrolidone, tetrahydrofuran, methyl tert-butyl ether, or dimethylsulfoxide, and a temperature of from about 20° C. to about 60° C., or from about 20° C. to about 25° C.

In one embodiment, provided is a process for preparing a compound of Formula (I) or a salt thereof:

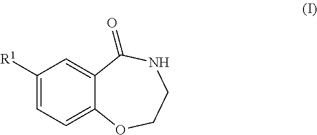

comprising contacting a compound of Formula (VIII) or a salt thereof:

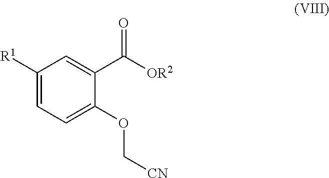

with a reducing agent, under reaction conditions sufficient to provide the compound of Formula (II) or a salt thereof,

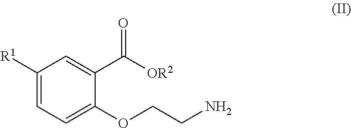

and cyclizing a compound of Formula (II) or a salt thereof to provide the compound of formula (I) or a salt thereof, wherein:

R¹ is hydrogen or halo; and

R² is hydrogen or alkyl optionally substituted with aryl.

In certain embodiments, the reducing agent is Raney Nickel and H₂, BH₃-tetrahydrofuran, BH₃-dimethyl sulfide, NaBH₄/CoCl₂, 5-ethyl-2-methyl-pyridine borane complex, lithium tri-t-butoxy aluminum hydride, sodium bis(2-methoxyethoxy)aluminumhydride, borane-N,N-diethyl aniline complex, diisobutylaluminium hydride or 9-borabicyclo[3.3.1]nonane. In some embodiments, the reaction conditions further comprise methanol, ethanol, isopropanol, tetrahydrofuran, or 2-methyltetrahydrofuran, and a temperature of from about 20° C. to about 50° C., or from about 20° C. to about 25° C. In some embodiments, the process is performed under pressure.

In one embodiment, provided is a process for preparing a compound of Formula (II) or a salt thereof:

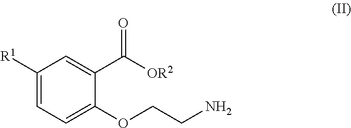

comprising contacting a compound of Formula (VIII) or a salt thereof:

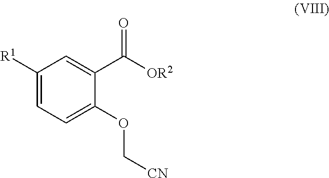

with a reducing agent under reaction conditions sufficient to provide the compound of Formula (II) or a salt thereof, wherein:

R¹ is hydrogen or halo; and

R² is hydrogen or alkyl optionally substituted with aryl.

In certain embodiments, the reducing agent is hydrogen gas. In certain embodiments, the reducing agent comprises an optional catalyst. The catalyst can be any suitable catalyst, such as palladium on carbon, platinum on carbon, or rhodium on carbon. The reaction may further comprising HCl, H₂SO₄, HBr, or H₃PO₄. In some embodiments, the reducing agent is borane-tetrahydrofuran, borane-dimethyl sulfide, or sodium borohydride. The reaction conditions may further comprise methanol, ethanol, or isopropanol.

In one embodiment, the compound of Formula (VIII) or a salt thereof:

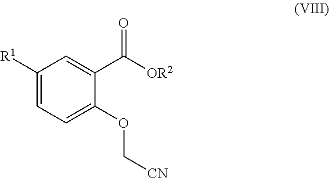

is prepared by contacting a compound of Formula (IV) with a compound of Formula XCH₂CN, where X is halo,

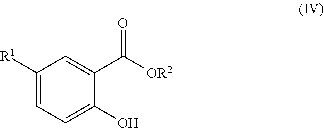

under reaction conditions sufficient to provide the compound of Formula (VIII) or a salt thereof, wherein:

R¹ is hydrogen or halo; and

R² is hydrogen or alkyl optionally substituted with aryl.

In certain embodiments, the reaction conditions comprise a base. In some embodiments, the base is K₂CO₃, Na₂CO₃, Cs₂CO₃, triethylamine, sodium hydride, or sodium hexamethyldisilazide. In certain embodiments, the reaction conditions further comprise dimethylacetamide, dimethylformamide, N-methyl-2-pyrrolidone, dimethylsulfoxide, tetrahydrofuran, or methyl tert-butyl ether, and a temperature of from about 20° C. to about 50° C., or from about 20° C. to about 25° C.

In one embodiment, R¹ is bromo. In another embodiment, X is Cl.

In one embodiment, provided is a process for preparing a compound of Formula (I) or a salt thereof:

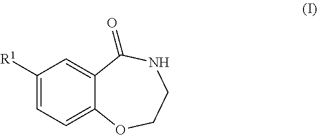

comprising contacting a compound of Formula (IX) or a salt thereof:

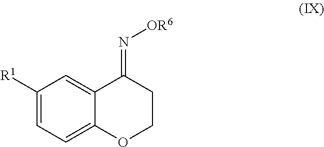

with an acid under reaction conditions sufficient to provide a compound of Formula (I) or a salt thereof, wherein:

R¹ is hydrogen or halo;

R⁶ is hydrogen or —S(O)₂R⁵; and

R⁵ is selected from the group consisting of alkyl, cycloalkyl, heterocyclyl, aryl, and heteroaryl, wherein each cycloalkyl, heterocyclyl, aryl, and heteroaryl is optionally substituted with one to three C₁₋₄ alkyl.

In certain embodiments, the acid is boron trichloride, boron trifluoride, boron tribromide, or polyphosphoric acid. In some embodiments, the reaction conditions further comprise dichloromethane, or toluene, and a temperature of from about 20° C. to about 100° C., or from about 20° C. to about 25° C.

In one embodiment, R¹ is bromo. In one embodiment, R⁶ is hydrogen. In another embodiment, R⁶ is —S(O)₂R⁵.

In certain embodiments, the reaction conditions comprise a base, such as pyridine, triethylamine or sodium acetate, for example. In some embodiments, the reaction conditions further comprise methanol, or ethanol, and a temperature of from about 20° C. to about 80° C., or about 75° C.

In one embodiment, the compound of Formula (IX) or a salt thereof:

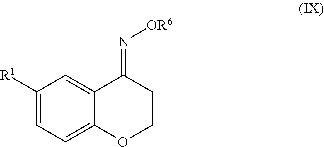

is prepared by contacting a compound of Formula (X) or a salt thereof:

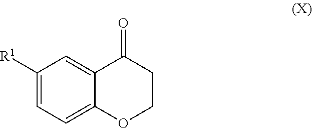

with hydroxylamine or hydroxylamine hydrochloride, optionally followed by a reagent of the formula X—S(O)₂R⁵, where X is halo, under reaction conditions sufficient to provide a compound of Formula (IX) or a salt thereof, wherein:

R¹ is hydrogen or halo;

R⁶ is hydrogen or —S(O)₂R⁵; and

R⁵ is selected from the group consisting of alkyl, cycloalkyl, heterocyclyl, aryl, and heteroaryl, wherein each cycloalkyl, heterocyclyl, aryl, and heteroaryl is optionally substituted with one to three C₁₋₄ alkyl.

In one embodiment, R¹ is bromo. In one embodiment, R⁶ is hydrogen. In another embodiment, R⁶ is —S(O)₂R⁵.

In certain embodiments, the reaction conditions comprise a base, such as pyridine, diisopropylethylamine or triethylamine, for example. In some embodiments, the reaction conditions further comprise methanol, or ethanol, and a temperature of from about −20° C. to about 20° C., or from about 0 to about 5° C.

In certain embodiments, the reagent of the formula X—S(O)₂R⁵ is methanesulfonyl chloride or toluenesulfonyl chloride.

In one embodiment, provided is a process for preparing a compound of Formula (I) or a salt thereof:

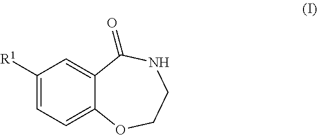

or a salt thereof, comprising contacting a compound of Formula (XI) or a salt thereof:

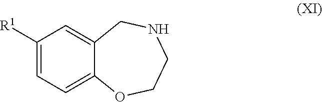

with an oxidant under reaction conditions sufficient to provide the compound of Formula (I) or a salt thereof, wherein:

R¹ is hydrogen or halo; and

R² is hydrogen or alkyl optionally substituted with aryl.

In some embodiments, the oxidant is manganese dioxide, N-bromosuccinimide, hydrogen peroxide, sodium chlorite, dihydrodicyanoquinone, or TEMPO. In certain embodiments, the reaction conditions further comprise DCM, methyl tert-butyl ether or tetrahydrofuran.

In one embodiment, the compound of Formula (XI) or a salt thereof:

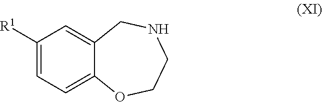

is prepared by contacting a compound of Formula (VIII) or a salt thereof:

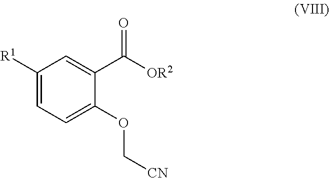

with a reducing agent under reaction conditions sufficient to form a compound of Formula (XI) or a salt thereof, wherein:

R¹ is hydrogen or halo; and

R² is hydrogen or alkyl optionally substituted with aryl.

In certain embodiments, the reducing agent is BH₃-dimethyl sulfide, BH₃-tetrahydrofuran, NaBH₄, or NaCNBH₄. Any suitable solvent can be used, such as tetrahydrofuran, 2-methyltetrahydrofuran, or methyl tert-butyl ether, and a temperature of between about 20 and about 80° C.

In another embodiment, provided is a process for preparing a compound of Formula (IA), or a salt thereof:

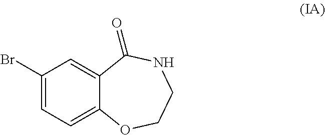 [SMILES: O=C1NCCOc2ccc(Br)cc21]

comprising contacting a compound of Formula (IB), or a salt thereof:

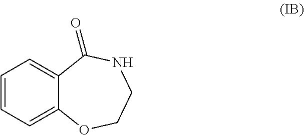 [SMILES: O=C1NCCOc2ccccc21]

with Br₂, under reaction conditions sufficient to provide a compound of Formula (IA), or a salt thereof.

In one embodiment, provided is a process for preparing a compound of Formula (XIIA), or a salt thereof:

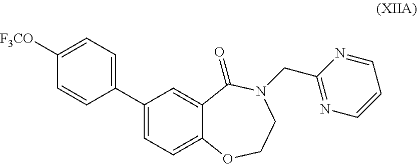 [SMILES: O=C1c2cc(-c3ccc(OC(F)(F)F)cc3)ccc2OCCN1Cc1ncccn1]

comprising the steps of:

a) contacting a compound of Formula (I), or a salt thereof:

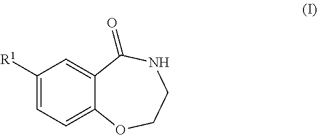

with a compound of the formula

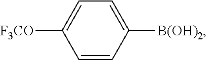 [SMILES: OB(O)c1ccc(OC(F)(F)F)cc1]

or a boronic ester thereof, under reaction conditions sufficient to provide a compound of Formula (IC), or a salt thereof; and

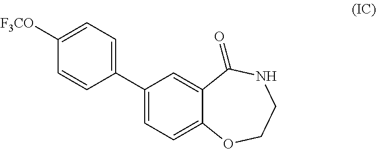 [SMILES: O=C1NCCOc2ccc(-c3ccc(OC(F)(F)F)cc3)cc21]

b) contacting the compound of Formula (IC), or a salt thereof, with a compound of the formula

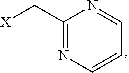

where X is halo, under reaction conditions sufficient to provide the compound of Formula (XIIA) or a salt thereof,

wherein:
R¹ is hydrogen or halo; and
R² is hydrogen or alkyl optionally substituted with aryl.

In one embodiment, the compound of Formula (I), or a salt thereof, is provided from any of the processes described herein.

In a specific embodiment, provided is a process for preparing a compound of Formula (XIIA), or a salt thereof:

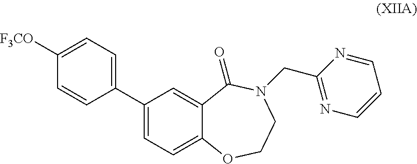 [SMILES: O=C1c2cc(-c3ccc(OC(F)(F)F)cc3)ccc2OCCN1Cc1ncccn1]

comprising the steps of:

a) contacting a compound of Formula (VA), or a salt thereof, with a compound of Formula (IVA), or a salt thereof;

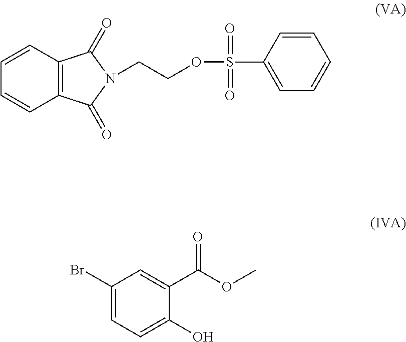 [SMILES: O=C1c2ccccc2C(=O)N1CCOS(=O)(=O)c1ccccc1 ; COC(=O)c1cc(Br)ccc1O]

in the presence of a base, under reaction conditions sufficient to provide the compound of Formula (IIIA) or a salt thereof;

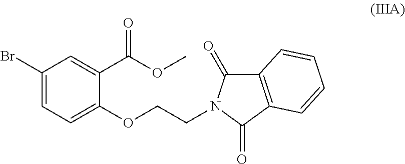 [SMILES: COC(=O)c1cc(Br)ccc1OCCN1C(=O)c2ccccc2C1=O]

b) deprotecting and cyclizing a compound of formula (IIIA) or a salt thereof, under reaction conditions sufficient to provide the compound of Formula (IA) or a salt thereof;

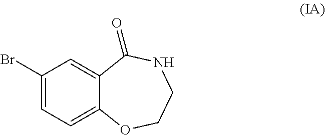 [SMILES: O=C1NCCOc2ccc(Br)cc21]

c) contacting a compound of Formula (IA), or a salt thereof, with a compound of the formula

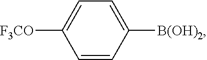 [SMILES: OB(O)c1ccc(OC(F)(F)F)cc1]

or a boronic ester thereof, under reaction conditions sufficient to provide a compound of Formula (IC), or a salt thereof; and

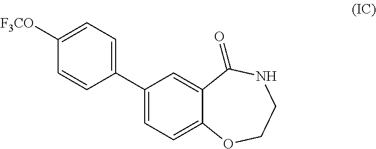 [SMILES: O=C1NCCOc2ccc(-c3ccc(OC(F)(F)F)cc3)cc21]

d) contacting the compound of Formula (IC), or a salt thereof, with a compound of the formula

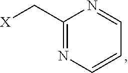

where X is halo, under reaction conditions sufficient to provide the compound of Formula (XIIA) or a salt thereof.

In one embodiment, provided is a process for preparing a compound of Formula (XII) or a salt thereof:

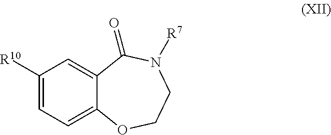

comprising the steps of:

a) cyclizing a compound of Formula (III) or a salt thereof, under reaction conditions sufficient to provide the compound of Formula (I) or a salt thereof:

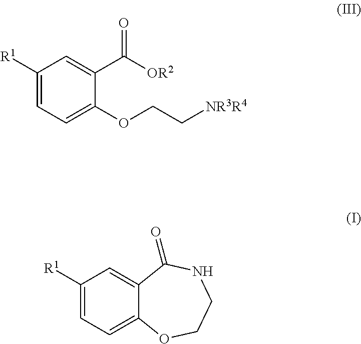

b) contacting the compound of Formula (I), or a salt thereof, with a compound of the formula X—R⁷, where X is halo or —S(O)₂R⁵, under reaction conditions sufficient to provide the compound of Formula (XII) or a salt thereof, wherein:

R¹ is hydrogen or halo;

R² is hydrogen or alkyl optionally substituted with aryl;

R³ is hydrogen or a nitrogen protecting group;

R⁴ is hydrogen, or R³ and R⁴ together with the nitrogen to which they are attached form N-diphenylmethyleneamine or a succinimide;

R⁵ is selected from the group consisting of alkyl, cycloalkyl, heterocyclyl, aryl, and heteroaryl, wherein each cycloalkyl, heterocyclyl, aryl, and heteroaryl is optionally substituted with one to three C₁₋₄ alkyl;

R⁷ is —C₁₋₆ alkylene-R⁸, -L-R⁸, -L-C₁₋₆ alkylene-R⁸, —C₁₋₆ alkylene-L-R⁸ or —C₁₋₆ alkylene-L-C₁₋₆ alkylene-R⁸;

L is —O—, —S—, —C(O)—, —NHS(O)₂—, —S(O)₂NH—, —C(O)NH— or —NHC(O)—, provided that when R⁷ is -L-R⁸ or -L-C₁₋₆ alkylene-R⁸, then L is not —O—, —S—, —NHS(O)₂— or —NHC(O)—;

R⁸ is cycloalkyl, aryl, heteroaryl or heterocyclyl; wherein said cycloalkyl, aryl, heteroaryl or heterocyclyl are optionally substituted with one, two or three substituents independently selected from the group consisting of C₁₋₆ alkyl, C₂₋₄ alkynyl, halo, —NO₂, cycloalkyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O—R²⁰; wherein said C₁₋₆ alkyl, cycloalkyl, aryl, heterocyclyl or heteroaryl are optionally further substituted with one, two or three substituents independently selected from the group consisting of halo, —NO₂, C₁₋₆ alkyl, cycloalkyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, cycloalkyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;

R¹⁰ is hydrogen, halo, aryl, cycloalkyl, cycloalkenyl, heterocyclyl, or heteroaryl, wherein each aryl, cycloalkyl, cycloalkenyl, heterocyclyl, or heteroaryl is optionally substituted with one to three R¹¹;

each R¹¹ is independently halo, hydroxyl, —NO₂, —CN, —CF₃, —OCF₃, —Si(CH₃)₃, C₁₋₄ alkyl, C₁₋₃ alkoxy, C₂₋₄ alkenyl, C₂₋₄ alkynyl, aralkyl, aryloxy, aralkyloxy, acyl, carboxy, carboxyester, acylamino, amino, substituted amino, cycloalkyl, aryl, heteroaryl and heterocyclyl;

when R²⁰ and R²² are attached to a common nitrogen atom R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₃ alkoxy, —CF₃, —OCF₃, aryl, heteroaryl and cycloalkyl; and

each R²⁶ is independently selected from the group consisting of hydrogen, C₁₋₄ alkyl, aryl and cycloalkyl; wherein the C₁₋₄ alkyl, aryl and cycloalkyl may be further substituted with from 1 to 3 substituents independently selected from the group consisting of hydroxyl, halo, C₁₋₄ alkoxy, —CF₃ and —OCF₃.

In one embodiment, R¹ is bromo. In one embodiment, R² is methyl. In some embodiments, R¹¹ is aryl, optionally substituted with —CF₃ or —OCF₃.

### 3. Compounds

In other embodiments, the disclosure provides for intermediate compounds that may be used in the processes described herein. Thus, for instance, one embodiment is a compound of the formula:

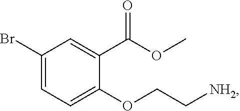

or a salt thereof. In certain embodiments, the compound is the HCl salt.

In another embodiment, provided is a compound of the formula:

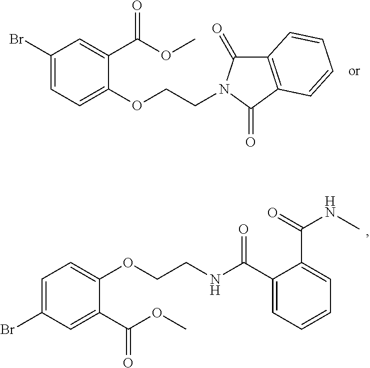 [SMILES: COC(=O)c1cc(Br)ccc1OCCN1C(=O)c2ccccc2C1=O ; CNC(=O)c1ccccc1C(=O)NCCOc1ccc(Br)cc1C(=O)OC]

or a salt thereof.

In yet another embodiment, provided is a compound of the formula:

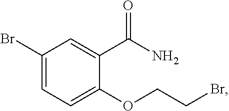 [SMILES: NC(=O)c1cc(Br)ccc1OCCBr]

or a salt thereof.

In still another embodiment, provided is a compound of the formula:

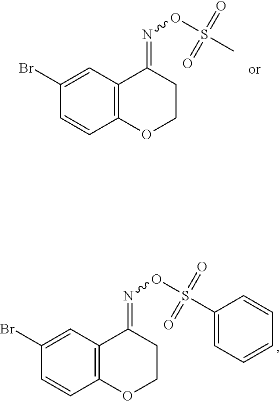 [SMILES: CS(=O)(=O)ON=C1CCOc2ccc(Br)cc21 ; O=S(=O)(ON=C1CCOc2ccc(Br)cc21)c1ccccc1]

or a salt thereof.

### EXAMPLES

The compounds of the disclosure may be prepared using methods disclosed herein and routine modifications thereof which will be apparent given the disclosure herein and methods well known in the art. Conventional and well-known synthetic methods may be used in addition to the teachings herein. The synthesis of compounds described herein, may be accomplished as described in the following examples. If available, reagents may be purchased commercially, e.g. from Sigma Aldrich or other chemical suppliers. Unless otherwise noted, the starting materials for the following reactions may be obtained from commercial sources.

### Example 1

### Process to Compound (IA)

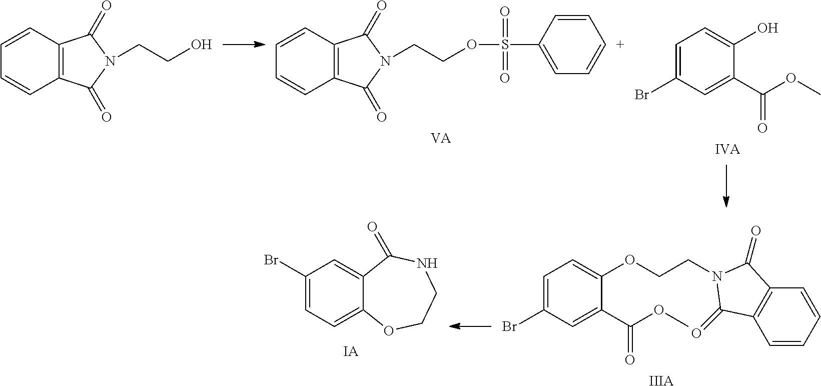

### Activation of 2-(2-hydroxyethyl)isoindoline-1,3-dione to Form VA

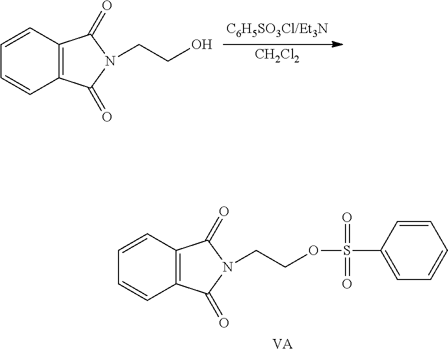

To the mixture of commercially available 2-(2-hydroxyethyl) isoindoline-1,3-dione (8.8 g, 1.00 equiv) and triethylamine (5.8 g, 1.25 equiv) in methylene chloride (69 mL) is added benzenesulfonyl chloride (9.3 g, 1.05 equiv) dropwise at under about 25° C. The mixture is stirred at room temperature until the reaction is complete as determined by HPLC. The reaction mixture is washed with an aqueous solution of sodium bicarbonate. The organic solution is concentrated under reduced pressure and the product is precipitated by adding hexanes (83 mL) to the residue. VA is isolated by filtration (15.1 g, 99% yield). ¹H NMR (400 MHz, DMSO-d₆): δ 7.77-7.82 (m, 4H), 7.71 (d, J=8.0, 2H), 7.52 (t, J=8.0, 1H), 7.41 (t, J=8.0, 2H), 4.29 (t, J=4.0, 2H), 3.81 (t, J=4.0, 2H).

However, alternative reagents and reaction conditions to those disclosed above may also be employed. For example, other aromatic sulfonate groups, halogens, or carbonates may be employed in lieu of benzenesulfonyl chloride. In addition, the nitrogen may be protected with another amine protecting group, such as tert-butyl carbamate (N-Boc), benzyl, allyl, or as an imine, such as N-diphenylmethyleneamine. Further, various organic bases (e.g., iPr₂NEt, DBU, DMAP), alkali metal bases (e.g., NaH), or hexamethyldisilazane bases (e.g., Na, K, LiHMDS) may be used. Alternative solvents may also be used, such as other organic solvents e.g., toluene, THF) or polar aprotic solvents (e.g., DMF, DMA), and temperatures ranging from about 0 to about 40° C. may be employed.

### Coupling of VA and IVA to IIIA

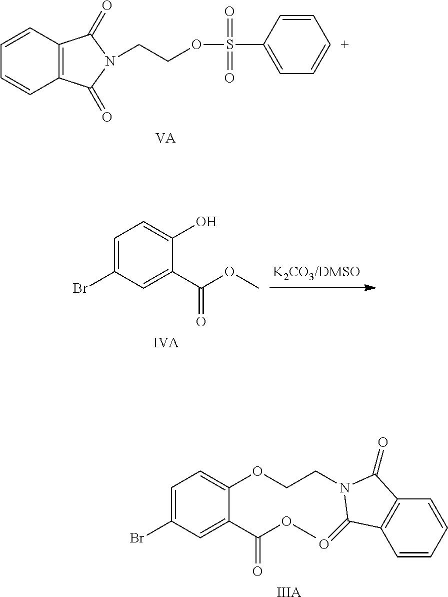

A mixture of IVA (9 g, 1.0 equiv) and VA (14.8 g, 1.15 equiv) in DMSO (54 mL) is charged K₂CO₃ (10.7 g, 2.0 equiv). The mixture is heated to 50 to 55° C. and monitored by HPLC until the reaction is complete. The mixture is cooled to about 30° C. and diluted with EtOAc (108 mL) and cooled further to 20° C. The pH is adjusted to pH 5-6 by the slow addition of concentrated HCl (13.5 g, CO₂ evolution and highly exothermic), maintaining the internal temperature at under about 30° C. The organic solution is washed with water (45 mL). The final organic solution is concentrated under reduced pressure to minimum volume. Hexanes (108 mL) is charged and the resultant slurry is agitated. The slurry is filtered and dried at about 50° C. under vacuum to afford 14.9 g IIIA (95% yield). ¹H NMR (400 MHz, DMSO-d₆): δ 7.81-7.88 (m, 4H), 7.62-7.65 (m, 2H), 7.12-7.14 (m, 1H), 4.28 (t, J=8.0, 2H), 3.95 (t, J=4.0, 2H), 3.56 (s, 3H).

However, alternative reagents and reaction conditions to those disclosed above may also be employed. For example, various bases, such as organic bases (e.g., iPr₂NEt, DBU, DMAP), alkali metal bases (e.g., NaH), hexamethyldisilazane bases (e.g., Na, K, LiHMDS), carbonate bases (e.g., Cs₂CO₃, Na₂CO₃), or alkoxides (e.g., potassium tert-butoxide) may be used. Alternative polar aprotic solvents may also be used, such as DMF, DMA, or NMP, and temperatures ranging from about 30 to about 75° C. may be employed.

### Phthalamide Deprotection of IIIA to IIA and Cyclization to IA

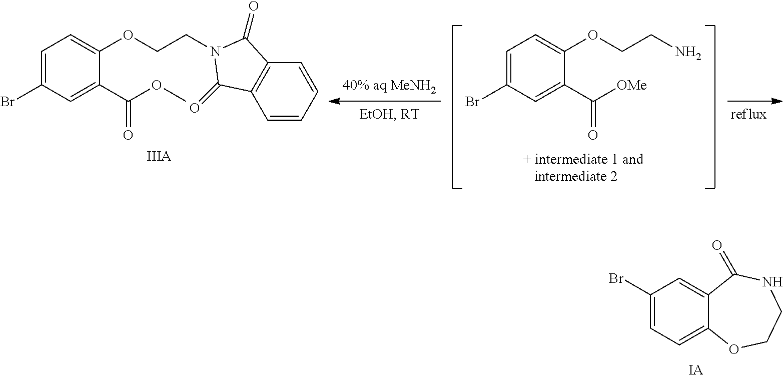

IIIA (13.7 g, 1.00 equiv) in EtOH (69 mL) is charged a 40% aqueous solution of MeNH₂ (8.8 mL, 3.00 equiv). The mixture is stirred at ambient temperature until most of the solids are dissolved and then heated to reflux (about 85° C.) and aged until the reaction is complete by HPLC analysis. The mixture is concentrated to minimum volume. Dichloromethane (96 mL) and an aqueous solution of NaOH (5 wt %, 53 mL) is charged and the mixture is agitated. The biphasic mixture is separated. To the organic layer is charged water (37 mL) and the pH is adjusted to pH 2-3 with concentrated HCl. The organic layer is washed twice with water (37 mL) and dried over Na₂SO₄. The mixture is filtered and the solution is concentrated under reduced pressure to a minimum volume. Hexanes (66 mL) is added and the slurry is agitated at about 25° C. for about 2 hours. The slurry is filtered and the solids are washed with hexanes (10 mL). The solids are dried under vacuum to afford 6.7 g of IA as a solid (82% yield). ¹H NMR for IA: (400 MHz, DMSO-d₆): δ 8.46 (s, 1H), 7.87 (d, J=4.0, 1H), 7.57 (dd, J=2.0, 8.0, 1H), 6.95 (d, J=8.0, 1H), 4.29 (t, J=4.0, 2H), 3.33 (dd, J=4.0, 8.0, 2H).

### Intermediate 1

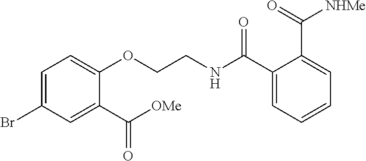 [SMILES: CNC(=O)c1ccccc1C(=O)NCCOc1ccc(Br)cc1C(=O)OC]

¹H NMR (400 MHz, DMSO) δ 8.34 (br t, J=5.0 Hz, 1H), 8.20 (br d, J=4.3 Hz, 1H), 7.80 (d, J=2.5 Hz, 1H), 7.70 (dd, J=8.9, 2.6 Hz, 1H), 7.49 (s, 4H), 7.20 (d, J=8.9 Hz, 1H), 4.19 (br t, J=5.2 Hz, 2H), 3.79 (s, 3H), 3.62 (br d, J=5.3 Hz, 2H), 2.71 (d, J=4.5 Hz, 3H). ¹³C NMR (100 MHz, DMSO) δ 168.98, 168.94, 165.37, 157.23, 136.67, 136.54, 136.34, 133.32, 129.85, 129.70, 128.12, 128.01, 122.94, 117.00, 112.12, 67.92, 52.60, 38.96, 26.53.

### Intermediate 2

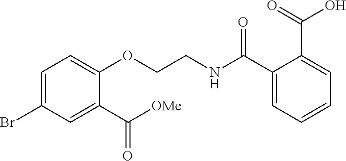 [SMILES: COC(=O)c1cc(Br)ccc1OCCNC(=O)c1ccccc1C(=O)O]

¹H NMR (400 MHz, dmso) δ 13.5-12.5 (br, 1H), 8.40 (t, J=5.6 Hz, 1H), 7.78 (dd, comp, 2H), 7.71 (dd, J=8.9, 2.6 Hz, 1H), 7.57 (td, J=7.5, 1.3 Hz, 1H), 7.51 (td, J=7.6, 1.3 Hz, 1H), 7.45-7.37 (m, 1H), 7.20 (t, J=8.8 Hz, 1H), 4.17 (t, J=6.1 Hz, 2H), 3.77 (s, 3H), 3.57 (q, J=5.9 Hz, 2H). ¹³C NMR (101 MHz, dmso) δ 168.92, 167.81, 164.91, 156.65, 138.34, 135.85, 132.79, 131.22, 130.55, 129.24, 127.54, 122.58, 116.50, 111.65, 67.16, 52.17, 38.36.

However, alternative reagents and reaction conditions to those disclosed above may also be employed. For example, other MeNH₂ derivatives such as Me₂N(CH₂)₃NH₂, may be used, or various other reagents, such as hydrazine or hydrazine derivatives, hydroxylamine or ethylenediamine. Other organic water miscible solvents (e.g., methanol, isopropyl alcohol, DMF, acetonitrile, 2-methyltetrahydrofuran, or iPrOAc, etc.) may also be used, and temperatures may range from about 60 to about 100° C.

### Example 2

### Alternate Processes for Preparing IA

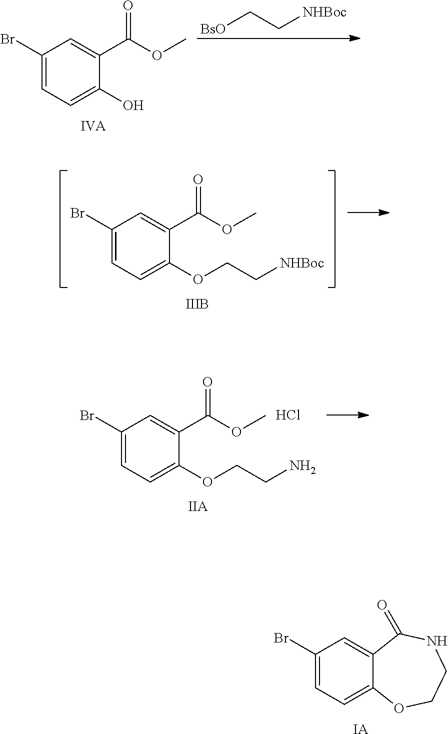

### Synthesis of IIA from IVA

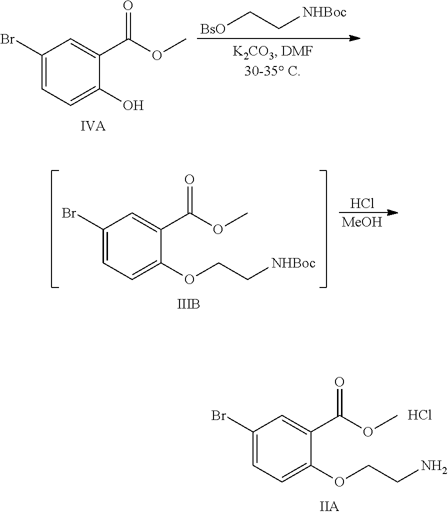

To a solution of 2-(tert-butoxycarbonylamino)ethyl benzenesulfonate (1.0 equiv) in DMF (5.4 vol) is charged IVA (0.9 equiv) and potassium carbonate (2.0 equiv). The mixture is heated to about 35° C. for about 24 hours and the reaction is monitored by HPLC until it is deemed complete. Upon reaction completion, the mixture is cooled to ambient temperature and toluene (3 vol) is charged. The mixture is cooled to about 20° C. and water (10.8 vol) is charged. The biphasic mixture is separated and the organic solution is washed twice with water (1.2 vol), followed by brine (0.5 vol). The organic solution is concentrated at about 50° C. to minimum volume. To a solution of IIIB (1.0 equiv) in methanol (1.6 vol) at ambient temperature is charged a solution of HCl in methanol (7.1-7.5 wt % solution, 3 equiv). The reaction is aged until the reaction is deemed complete. The reaction mixture is concentrated at about 45° C. until a thick slurry is formed. MTBE (4.7 vol) is charged and the slurry is agitated for 2 hours. The slurry is filtered and the filter cake is washed with MTBE (1 vol). The product is dried under vacuum at about 35° C. to provide IIA as the HCl salt (typical purity is >99% AN). ¹H NMR (400 MHz, dmso) δ 8.25 (s, 3H), 7.81 (d, J=2.6 Hz, 1H), 7.74 (dd, J=8.9, 2.6 Hz, 1H), 7.22 (d, J=8.9 Hz, 1H), 4.28 (t, J=5.3 Hz, 2H), 3.82 (s, 3H), 3.19 (s, 2H). ¹³C NMR (101 MHz, dmso) δ 164.69, 156.17, 136.07, 132.94, 122.75, 117.34, 112.45, 66.07, 52.34, 38.11.

However, alternative reagents and reaction conditions to those disclosed above may also be employed. For example, for the O-alkylation, other carbonate bases (i.e. Na₂CO₃, Cs₂CO₃) or organic bases (i.e. Et₃N) or metal bases (i.e. NaH, sodium hexamethyldisilazane) may be used. Alternative solvents may also be used, such as DMSO, NMP, DMA, or THF, and temperatures ranging from about 20 to about 50° C. may be employed. In addition, for the deprotection, other strong bronsted acids, such as H₃PO₄, H₂SO₄, trifluoroacetic acid, or toluenesulfonic acid, may be used. Alternative solvents may also be used, such as other alcoholic solvents (e.g., ethanol, or isopropanol) or organic solvents (e.g., MTBE, THF, or acetic acid).

### Cyclization of IIA to IA

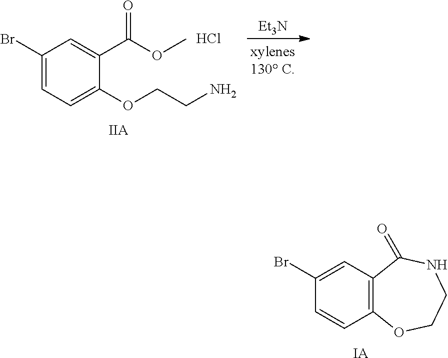

IIA (1.0 equiv), xylenes (5 vol), and triethylamine (2.0 equiv) is combined at ambient temperature and heated to about 130° C. The reaction progress is monitored by HPLC. Upon reaction completion, the reaction mixture is cooled to room temperature and dichloromethane (10 vol) and water (2 vol) are charged. The pH of the mixture is adjusted to pH 2 by the addition of aqueous HCl (6 M, ˜0.1 S). The biphasic mixture is separated and the aqueous layer is extracted with dichloromethane (1 vol). The combined organic solution is washed with water (2 vol) and brine (2 vol). The organic solution is treated with charcoal (0.1 S) and the slurry is filtered. The filter cake is washed with dichloromethane (1.5 vol) and the filtrate is concentrated until distillation stops. Hexanes (6.6 vol) is charged and the resultant slurry is aged, filtered, and dried in a vacuum oven at about 40° C. to provide IA as a solid.

However, alternative reagents and reaction conditions to those disclosed above may also be employed. For example, other salts may be formed and used in subsequent steps, such as the sulfate, phosphate, trifluoroacetate, or tosylate salt. Other bases may be employed, such as other organic bases (e.g., iPr₂NEt, or DBU) or metal bases (e.g., NaH, or sodium hexamethyldisilazane). Further, other high boiling solvents (e.g., toluene, or benzene), and temperatures ranging from about 95 to about 150° C. may be used.

### Example 3

### Alternate Route 2

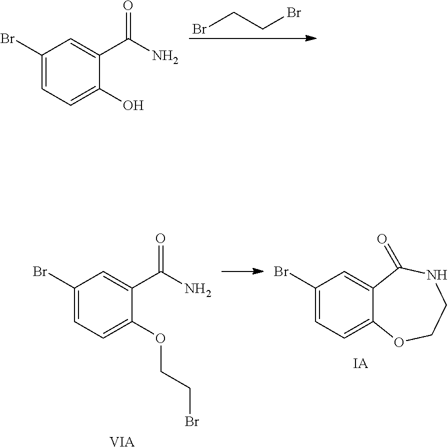

### Alkylation of 5-Bromosalicylamide to VIA

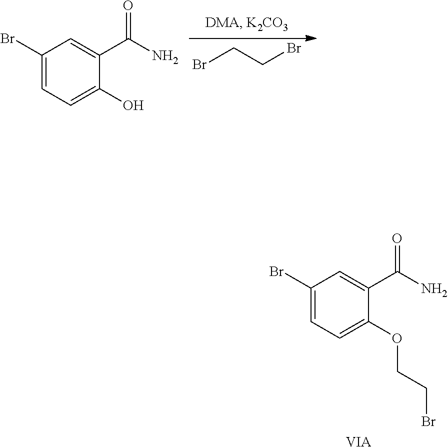

Combine 5-bromosalicylamide (1.0 g; 4.6 mmole), and DMA (10 ml) followed by addition of K₂CO₃ (1.9 g, 3 eq.) and 1,2-dibromoethane (0.8 ml, 2 eq.). The reaction mixture was stirred and checked by LCMS for reaction completion. The solids were removed via filtration followed by a rinse with iPrOAc (20 ml). The filtrate was washed with water (20 ml), 1M aq. HCl (10 ml) followed by brine (10 ml), and the organic layer was concentrated to dryness under vacuum. This residue was purified by silica gel chromatography to afford VIA (522 mg) as a solid. ¹H NMR (300 MHz, CDCl₃): δ=3.75 (t, J=5.3, 2H), 4.42 (t, J=5.3, 2H), 6.65 (brs, 1H), 6.80 (d, J=9.4, 1H), 7.52 (dd, J=9.4 2.3, 1H), 7.73 (brs, 1H) and 8.30 (d, J=2.3, 1H); ¹³C NMR (75 MHz, CDCl₃): δ=29.2, 68.6, 114.0, 114.4, 123.0, 135.3, 135.8, 155.2 and 165.6; LCMS: m/z (%)=321.8 (50), 323.8 (100) and 325.8 (50).

However, alternative reagents and reaction conditions to those disclosed above may also be employed. For example, other carbonate bases (e.g., Na₂CO₃, or Cs₂CO₃), organic bases (e.g., triethylamine) or metal bases (e.g., NaH, or sodium hexamethyldisilazane) may be used. Alternative solvents may also be used, such as other polar aprotic solvents (e.g., DMF, NMP, or DMSO) or ethereal solvents (e.g., THF, or MTBE) depending on the base, and temperatures may range from about 20 to about 60° C. depending on choice of solvent.

Cyclization of VIA4 to IA

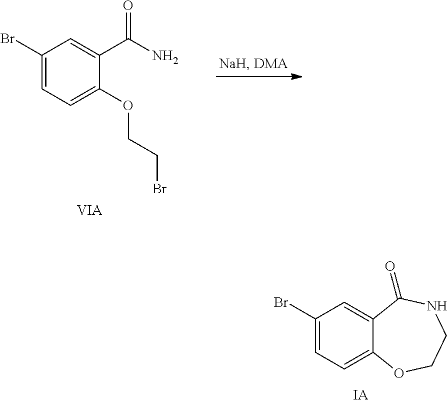

To a suspension of NaH (140 mg; 60% in mineral oil, 1 eq.) in DMA (2.5 ml) was slowly added a solution of VIA (0.9 g) in DMA (2.5 ml) while maintaining the internal temperature at less than 40° C. The resulting solution was stirred and checked by LCMS for reaction completion. At this point 1M aq. HCl (10 ml) was added followed by extraction with iPrOAc (10 mL). The organic layer was washed with 1M aq. HCl (10 ml) and brine (10 ml), sequentially, followed by drying over MgSO₄ and concentrated to dryness under vacuum. The residue was purified by silica gel chromatography to afford IA (258 mg) as a solid.

However, alternative reagents and reaction conditions to those disclosed above may also be employed. For example, other metal bases (e.g., sodium hexamethyldisilazane) may be used. Other polar aprotic solvents (e.g., DMF, NMP, or DMSO) and temperatures ranging from about −10 to about 40° C. may be employed.

### Example 4

### Alternate Route 3

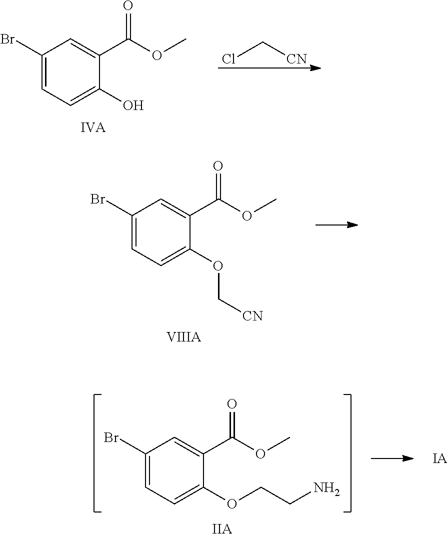

Alkylation of IVA to Form VIIIA

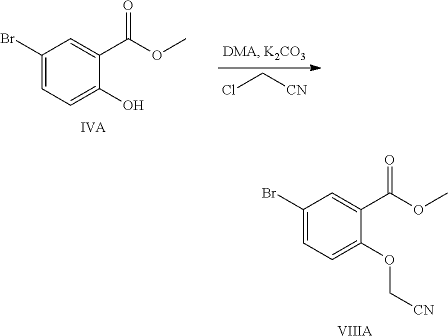

5-Bromosalicylic acid methyl ester IVA (5.0 g) in DMA (50 ml) was added K₂CO₃ (4.5 g, 1.5 eq.) and chloroacetonitrile (1.7 ml, 1.25 eq.). The resulting suspension was stirred overnight and checked by LCMS for reaction completion. The solids were removed via filtration followed by a rinse with iPrOAc (100 ml). The filtrate was washed with water (100 ml), 1M aq. HCl (50 ml) and water (50 ml), and the organic layer was dried over MgSO₄, treated with activated charcoal (Darco G60) (250 mg) followed by concentration to dryness under vacuum to afford VIIIA (5.2 g) as a solid. A small sample of this material (100 mg) was taken up in hot heptanes and the resulting solution was decanted from an orange oily residue. Upon cooling of the clear colorless solution of VIIIA (50 mg) was isolated as a solid. ¹H NMR (400 MHz, CDCl₃): δ=3.90 (s, 3H), 4.84 (s, 2H), 7.22 (d, J=8.6, 1H), 7.63 (dd, J=8.3, 2.3, 1H) and 7.98 (d, J=2.3, 1H); ¹³C NMR (100 MHz, CDCl₃): δ=52.6, 55.9, 114.7, 116.4, 118.5, 123.9, 134.9, 136.5, 155.2 and 164.3; LCMS: m/z (%)=270.0 (100) and 272.0 (100).

However, alternative reagents and reaction conditions to those disclosed above may also be employed. For example, alternative alkylating agents may be used, such as other haloacetonitriles (i.e., bromoacetonitrile or iodoacetonitrile) as well as aryl sulfonate compounds. In addition, other carbonate bases (e.g., Na₂CO₃, or Cs₂CO₃), organic bases (e.g., triethylamine) or metal bases (e.g., NaH, or sodium hexamethyldisilazane) may be used. Other polar aprotic solvents (e.g., DMF, NMP, or DMSO) or ethereal solvents (e.g., THF, or MTBE) and temperatures ranging from about 20 to about 50° C. may be employed.

### Nitrile Reduction and Cyclization of VIIIA to IA

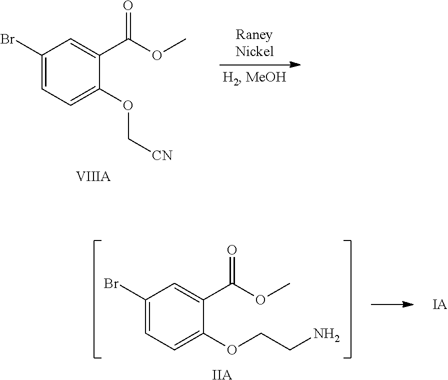

To a pressure flask was charged VIIIA (1.174 g), MeOH (10 ml), saturated aq. NH₃ (1 ml) and Raney-Nickel suspension (˜0.5 ml). The pressure flask was filled with H₂ three times. The resulting suspension was stirred under about 55 PSI H₂. The catalyst was removed via filtration followed by a rinse with MeOH. The filtrate was concentrated to dryness under vacuum. The residue was purified by amino functionalized silica gel chromatography using a gradient of 1% to 100% EtOAc in hexanes. The product containing fractions were pooled and concentrated to dryness to afford IA (220 mg) as a solid.

However, alternative reagents and reaction conditions to those disclosed above may also be employed. For example, alternative reducing agents may be used, such as borane-based reagents (e.g., BH₃-THF, BH₃-dimethyl sulfide), NaBH₄/CoCl₂, 5-ethyl-2-methyl-pyridine borane complex, LiAlH(OtBu)₃, Red-Al, Borane-N,N-diethyl aniline complex, DIBAL-H, or 9-BBN. In addition, other polar protic solvents (e.g., EtOH, or isopropanol) or ethereal solvents (e.g., THF, or 2-MeTHF) may be used depending on the reducing agent, lower or higher pressures of H₂ may be used (may impact on reaction rate) and temperatures may range from about 20 to about 50° C.

### Example 5

### Alternate Route 4

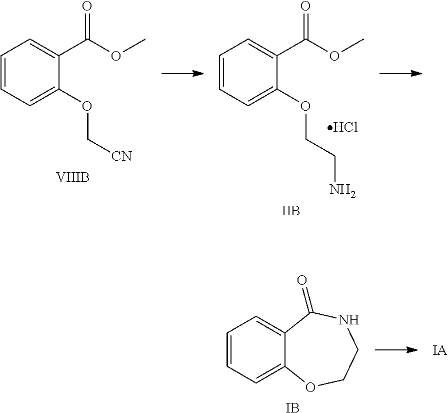

### Reduction of VIIIB to IIB

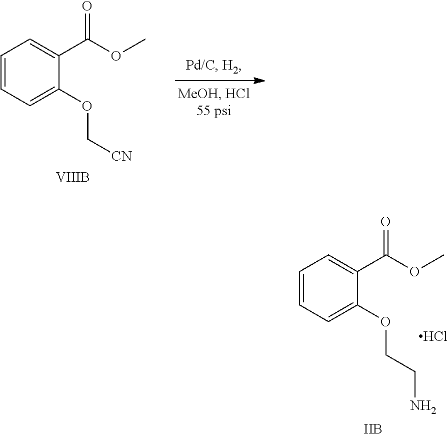

To a pressure flask was charged VIIIB (3.0 g), MeOH (30 ml), conc. aq. HCl (3 ml, 2 eq.) and 10% Pd/C (50% wet, 150 mg). The resulting suspension was evacuated and refilled with H₂ followed by stirring under about 55 PSI H₂ and monitored by LCMS and HPLC. Upon completion, the catalyst was removed via filtration followed by rinses with MeOH. The filtrate was concentrated to dryness under vacuum. The residue was taken up in MeCN and concentrated to dryness again under vacuum. This afforded IIB HCl salt (3.9 g) as a solid. ¹H NMR (300 MHz, DMSO-d₆): δ=3.18 (m, 2H), 4.27 (t, J=5.3 Hz, 2H), 7.07 (dd, J=8.2, 7.4 Hz, 1H), 7.20 (d, J=8.2 Hz, 1H), 7.54 (ddd, J=8.2, 7.7, 1.8 Hz, 1H), 7.67 (dd, J=7.7, 1.8 Hz, 1H) and 8.33 (brm, 3H); ¹³C NMR (75 MHz, DMSO-d₆): δ=38.7, 52.5, 66.2, 115.5, 121.2, 121.8, 131.4, 134.3, 157.4 and 166.6; LCMS: m/z (%)=196 (60), 164 (100).

However, alternative reagents and reaction conditions to those disclosed above may also be employed. For example, other heterogeneous catalysts (e.g., Pt/C, or Rh/C), other reducing agents (e.g., BH₃-THF or BH₃-dimethyl sulfide, or NaBH₄, and/or additives, such as other bronsted acids (e.g., H₂SO₄, HBr, or H₃PO₄) may be used. In addition, other polar protic solvents (e.g., EtOH, or isopropanol) or lower or higher pressures of H₂ may be employed.

### Cyclization of IIB to IB

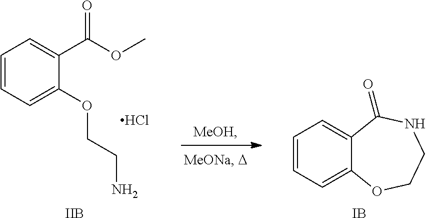

To a solution of IIB HCl salt (2.75 g, 11.9 mmole) in MeOH (27.5 ml) was added 30 wt % MeONa in MeOH (2.7 ml, 23.7 mmole). The resulting suspension was stirred at about 65° C. and the reaction was monitored by LCMS. The reaction mixture was cooled to ambient temperature and diluted with iPrOAc (55 ml) followed by filtration and a rinse with iPrOAc. The filtrate was reduced in volume under vacuum to dryness. The resulting suspension was filtered through a silica gel and rinsed with iPrOAc. The filtrate was concentrated to dryness under vacuum to afford IB (814 mg) as a solid. ¹H NMR (400 MHz, CDCl₃): δ=3.49 (m, 2H), 4.39 (t, J=4.9 Hz, 2H), 7.02 (d, J=8.2 Hz, 1H), 7.1 3 (dd, J=8.2, 7.4 Hz, 1H), 7.43 (dd, J=7.8, 7.4 Hz, 1H), 7.94 (d, J=7.8 Hz, 1H) and 8.38 (brm, 1H); ¹³C NMR (100 MHz, CDCl₃): δ=41.3, 73.4, 121.3, 122.8, 124.1, 131.6, 133.4, 155.3 and 171.2; LCMS: m/z (%)=164 (100).

However, alternative reagents and reaction conditions to those disclosed above may also be employed. For example, other carbonate bases (e.g., Na₂CO₃, or Cs₂CO₃) or organic bases (e.g., pyridine, or iPr₂NEt) may be used. In addition, other polar aprotic solvents (e.g., DMF, or DMA) or ethereal solvents (e.g., THF, or 2-MeTHF) depending on the choice of base and lower or higher temperatures may be used depending on choice of solvent.

### Bromination of IB to IA

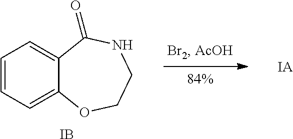

To a solution of IB (813 mg, 5.0 mmole) in AcOH (4 ml) was added Br₂ (282 μl, 5.5 mmole). The reaction mixture was stirred and monitored for reaction completion by LCMS. Water (20 ml) was then added and the resulting suspension was stirred. The solids were collected via filtration and rinsed with water followed by drying at about 60° C. in a vacuum oven to constant weight. This crude IA (1.268 g, 105%) solid was then subjected to purification by silica gel chromatography. The product containing fractions were pooled and concentrated to dryness under vacuum to afford IA (1.02 g) as a solid.

However, alternative reagents and reaction conditions to those disclosed above may also be employed. For example, other bromine sources, such as N-bromosuccinimide, Py₃HHBr, or dibromodimethylhydantoin, may be used. In addition, other mineral acids (i.e. H₂SO₄, TFA) solvents (e.g., DMF, or DMA) or ethereal solvents (e.g., THF, or 2-MeTHF) depending on the choice of base and temperatures ranging from about 0 to about 40° C. may be employed.

### Example 6

### Alternate Route 5

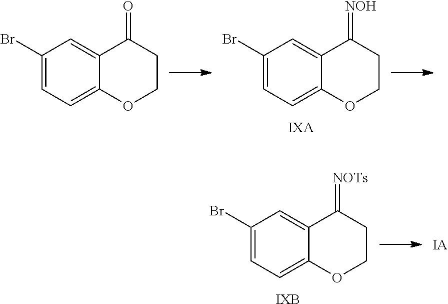

### Oxime Formation to IXA

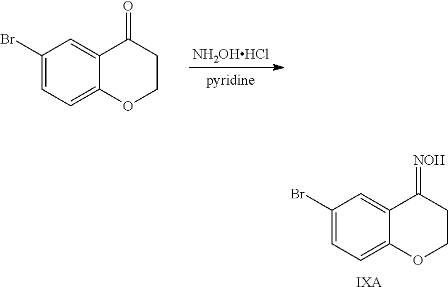

To a solution of hydroxylamine HCl (6.67 g; 96 mmole) in pyridine (80 ml) was added 6-bromochroman-4-one (9.08 g; 40 mmole). The reaction was stirred at about 75° C. was and monitored by HPLC for reaction completion. The reaction mixture was cooled to ambient and diluted with EtOAc (250 ml) and water (650 ml). This was mixed well and the organic layer was separated. The aqueous layer was extracted with EtOAc (100 ml). The organic layers were combined and washed twice with 20% aq. NaHSO₄ (300 ml each) and twice with brine (50 ml each) followed by drying over Na₂SO₄. The solution was concentrated to dryness under vacuum to afford IXA (9.88 g) as a solid. ¹H NMR (300 MHz, CDCl₃): δ=2.99 (t, J=6.2 Hz, 2H), 4.24 (t, J=6.2 Hz, 2H), 6.80 (d, J=8.8 Hz 1H), 7.34 (dd, J=8.8 Hz, 2.3 Hz, 1H) and 7.41 (d, J=2.3 Hz, 1H); ¹³C NMR (75 MHz, CDCl₃): δ=23.2, 65.0, 114.0, 119.7, 119.9, 126.7, 133.9, 149.1 and 155.6; LCMS: m/z (%)=241.9 (100) and 243.9 (100).

However, alternative reagents and reaction conditions to those disclosed above may also be employed. For example, other bases, such as triethylamine or NaOAc, may be used. In addition, other polar protic solvents (e.g., MeOH, or EtOH) and temperatures ranging from about 20 to about 80° C. may be employed.

### Tosylation of IXA to IXB

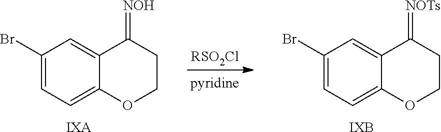

To a solution of IXA (1.21 g; 5 mmole) in pyridine (5 ml) was added p-toluenesulfonyl chloride (1.24 g, 6.5 mmole). The reaction mixture was stirred and monitored for reaction completion by HPLC. Water (10 ml) was then added and the resulting suspension was stirred at about 0° C. The solids were obtained via filtration and washed with water (10 ml) followed by drying in a vacuum oven to afford IXB (2.0 g) as a solid. ¹H NMR (300 MHz, CDCl₃): δ=2.45 (s, 3H), 2.97 (d, J=6.5 Hz, 2H), 4.19 (d, J=6.5 Hz, 2H), 6.78 (d, J=8.7 Hz, 1H), 7.37-7.41 (m, 3H), 7.87 (d, J=2.3 Hz, 1H) and 7.93 (d, J=8.2 Hz, 2H); ¹³C NMR (75 MHz, CDCl₃): δ=21.8, 24.6, 64.4, 114.0, 117.3, 119.9, 127.5, 129.0, 129.8, 132.2, 136.0, 145.5, 155.8 and 156.7; LCMS: m/z (%)=395.9 (40) and 397.9 (40), 223.9 (90) and 225.9 (90), 155 (100).

However, alternative reagents and reaction conditions to those disclosed above may also be employed. For example, alternative reagents, such as methanesulfonyl chloride and/or other bases, such as iPr₂NEt, or Et₃N, may be used. In addition, temperatures may range from about −20 to about 20° C.

### Rearrangement of IXB to IA

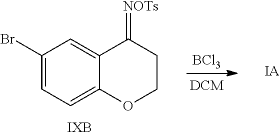

To a solution of IXB (100 mg, 0.25 mmole) in DCM (2 ml) was added 1M BCl₃ in toluene (0.75 ml, 0.75 mmole). The reaction was monitored for completion by HPLC analysis. Saturated aq. NaHCO₃ was then added until the pH was approximately 9. The aqueous layer was extracted twice with DCM (2×20 ml). The organic layers were combined and washed with brine (2×20 ml) and dried over Na₂SO₄. The resulting solution was concentrated to dryness under vacuum. The residue was purified by silica gel chromatography to afford IA as a solid.

However, alternative reagents and reaction conditions to those disclosed above may also be employed. For example, other acids, such as boron trifluoride, boron tribromide, or polyphosphoric acid, may be used, in addition to other suitable solvents, such as toluene. Temperatures may range from about 20 to about 100° C. depending on the acid used.

### Example 7

### Alternate Route 6

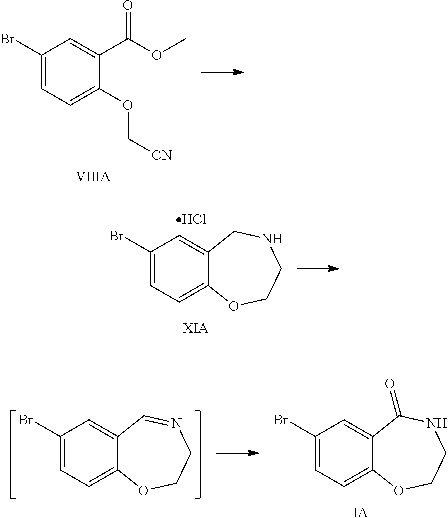

### Reduction and Cyclization of VIA to XIA

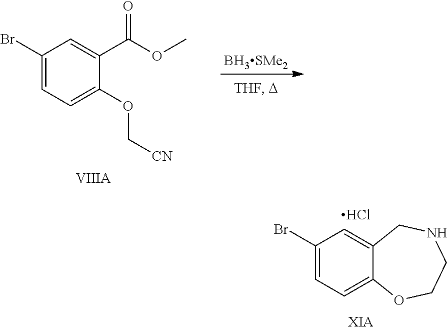

To a solution of VIIIA (2.9 g, 10.8 mmole) in THF (15 ml) was added 1M BH₃ in DMS (43 ml, 43 mmole). The resulting solution was stirred at reflux under the reaction is deemed complete by HPLC analysis. After cooling to ambient temperature, MeOH (6 ml, 148 mmole) was added slowly which resulting in off-gassing. Next 3M HCl in cyclopentylmethyl ether (60 ml, 180 mmole) was added and the resulting suspension was stirred. The solids were obtained via filtration and dried in a vacuum oven at about 40° C. to afford the HCl salt of XIA as a solid. ¹H NMR (300 MHz, DMSO-d6): δ=3.17 (t, J=5.0 Hz, 2H), 3.42 (brs, 3H), 4.16 (t, J=5.0 Hz, 2H), 6.91 (d, J=8.8 Hz, 1H), 7.36 (dd, J=8.8 and 2.4 Hz, 1H) and 7.47 (d, J=2.4 Hz, 1H); ¹³C NMR (75 MHz, DMSO-d6): δ=38.7, 58.0, 65.0, 113.0, 113.9, 130.0, 130.4, 134.2 and 154.3; LCMS: m/z (%)=228.0 (100) and 230.0 (100).

However, alternative reagents and reaction conditions to those disclosed above may also be employed. For example, other reducing agents, such as BH₃-THF, NaBH₄, or NaCNBH₄, may be used, in addition to other suitable solvents, such as 2-MeTHF, or MTBE. Temperatures may range from about 20 to about 80° C. depending on the solvent.

### Oxidation of XIA to IA

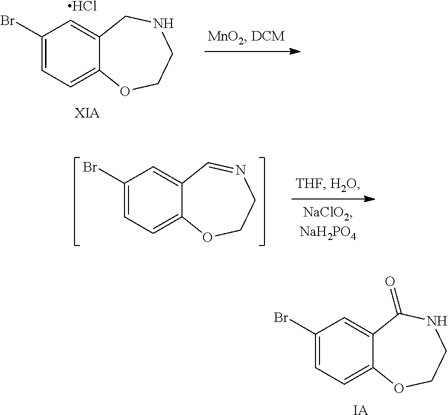

To a suspension of XIA HCl salt (1.14 g, 4.3 mmole) in DCM ((11 ml) was added 1M aq. KOH (11 ml, 11 mmole). This mixture was stirred until all the solids were dissolved followed by separation of the layers. The DCM layer was dried over MgSO₄ followed by the addition of MnO₂ (11.4 g, 131 mmole). The resulting suspension was stirred and monitored by LCMS. At this point the reaction was deemed complete and the solids were removed via filtration followed by a rinse with DCM. A small sample of the filtrate was concentrated to dryness for analysis. The bulk of the filtrate was solvent swapped into THF under vacuum. To the resulting THF solution of 7-bromo-2,3-dihydrobenzo[f][1,4]oxazepine was added 2-methyl-2-butene (4.6 ml, 43 mmole) followed by a solution of NaClO₂ (1.94 g, 21.5 mmole) in 1M aq. NaH₂PO₄ (6.5 ml, 6.5 mmole). The reaction mixture was stirred and checked by LCMS. Upon reaction completion, the reaction mixture was diluted with EtOAc and washed twice with 10% aq. Na₂S₂O₃ and once with brine. The resulting EtOAc solution was dried over MgSO₄ and concentrated to dryness under vacuum. The residue was purified by silica gel chromatography to afford IA as a solid.

However, alternative reagents and reaction conditions to those disclosed above may also be employed. For example, other oxidants, such as N-bromosuccinimide, hydrogen peroxide, sodium chlorite, dihydrodicyanoquinone, or TEMPO, may be used, in addition to other suitable solvents, such as THF, or MTBE.

### Example 8

### Synthesis of XIIA

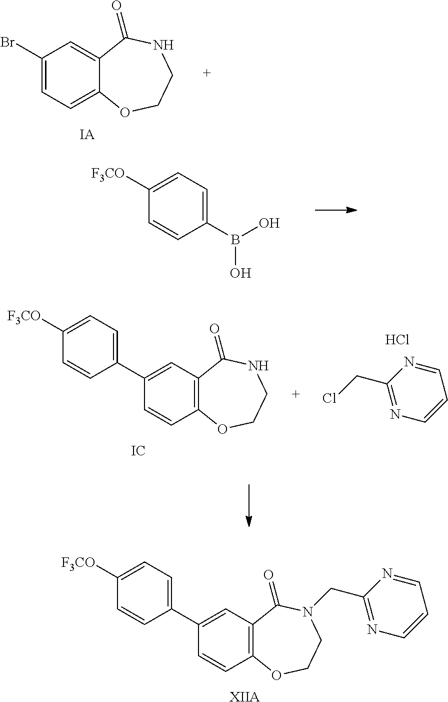

### Suzuki Coupling to IC

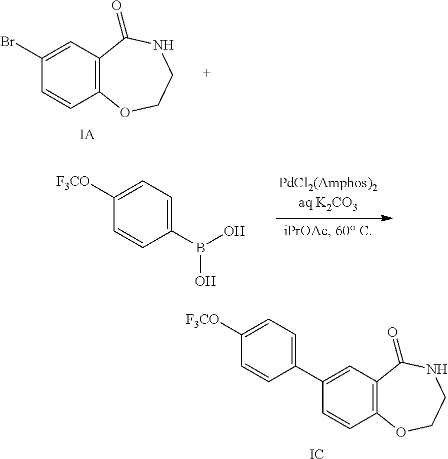

To a reactor are charged IA (100 g, 1.0 equiv) and (4-(trifluoromethoxy) phenyl)boronic acid (89.3 g, 1.05 equiv). The contents are inerted and a solution of degassed isopropyl acetate (1000 mL) and degassed aqueous potassium carbonate (165.6 g, 2.4 M aqueous solution) are charged. PdCl₂(Amphos)₂ (2.9 g, 0.01 equiv) is then charged and the contents are inerted. The heterogeneous mixture is heated to about 60° C. and agitated until the reaction is complete by HPLC analysis. Upon reaction completion, the mixture is cooled to about 45° C. and the phases are separated. The organic solution is washed with 1 wt % aqueous NaOH (500 mL) followed by 1 wt % aqueous NaCl (2×500 mL). The organic solution is concentrated under reduced pressure to approximately 400 mL, at which point the mixture becomes heterogeneous. The mixture is agitated and heated to about 55° C. and is charged n-heptane (1.2 L) is charged slowly. The slurry is slowly cooled to about −10° C., filtered, and dried to provide IC. ¹H NMR (400 MHz, DMSO-d₆): δ 8.43 (t, J=8.0, 1H), 8.05 (d, J=2.4, 1H), 7.72-7.76 (m, 3H), 7.41 (dd, J=1.0, 8.0, 2H), 7.09 (d, J=8.0, 1H), 4.32 (t, J=4.0, 2H), 3.30-3.37 (m, 2H).

However, alternative reagents and reaction conditions to those disclosed above may also be employed. For example, other catalysts may be used. Suitable catalysts include a combination of a metal (e.g., palladium) and a ligand (e.g., 1,1′-bis(diphenylphosphino)ferrocene]palladium, di-tert-butyl(4-dimethylamino)phenyl)phosphine, triphenylphosphine, tricyclohexylphosphine, tri-tert-butylphosphine, or a preformed metal/ligand complex such as 1,1′-bis(diphenylphosphino)ferrocene]palladium, bis(di-tert-butylphenyl)phosphine)dichloro-palladium. In addition, bases, such as carbonate or phosphate bases (e.g., sodium, lithium, cesium carbonate, or potassium phosphate), organic bases (e.g., NaOtBu, or NaOEt), hydroxide bases (e.g., NaOH, KOH, or CsOH), or fluoride bases (e.g., KF), may be employed. Various solvents and co-solvents may be used. For example, toluene, t-amyl alcohol, isopropyl alcohol, 2-methyltetrahydrofuran, or dioxane may be combined with from about 3 to about 7 volumes water. Temperatures may range from about 40 to about 80° C.

### Alkylation to XIIA

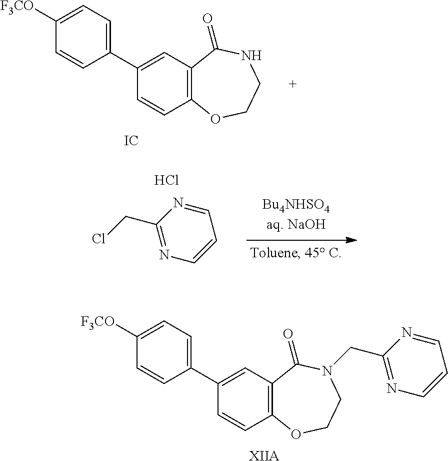

To a suspension of IC (50 g, 1.0 equiv), 2-(chloromethyl)pyrimidine hydrochloride (26.5 g, 1.2 equiv), Bu₄NHSO₄ (5.3 g, 0.1 equiv) in toluene (300 mL) was slowly charged a solution of 25 wt % aqueous NaOH (200 mL) at a rate such that the internal temperature is below 30° C. The heterogeneous mixture is warmed to about 45° C. and agitated until the reaction was deemed complete by HPLC analysis. Upon reaction completion, the reaction mixture was diluted with toluene (200 mL) and cooled to about 20° C. The biphasic mixture was separated and the organic solution was washed with 10 wt % brine (3×250 mL). The organic solution is concentrated under reduced pressure to about 200 mL. N-heptane (250 mL) is charged until the mixture becomes cloudy. The slurry is aged and, additional n-heptane (350 mL) is added slowly over a period of 1-2 hours. The mixture is cooled slowly to about 0° C. (−5 to 5° C.), filtered, and dried to provide IC. ¹H NMR (400 MHz, DMSO-d₆): δ 8.78 (d, J=4.8, 2H), 7.99 (d, J=2.4, 1H), 7.80 (dd, J=8.4, 2.4, 1H), 7.76 (dd, J=6.8, 2.4, 2H), 7.42 (d, J=8.8, 2H), 7.41 (t, J=4.8, 1H), 7.15 (d, J=8.4, 1H), 5.00 (s, 2H), 4.53 (t, J=4.4, 2H), 3.78 (t, J=4.8, 2H). ¹³C NMR (100 MHz, DMSO-d₆): δ 167.21, 166.29, 157.50, 154.00, 147.70, 138.26, 133.00, 131.20, 129.43, 128.20, 125.86, 122.05, 121.43, 121.38, 119.87, 72.90, 53.52, 47.84.

However, alternative reagents and reaction conditions to those disclosed above may also be employed. For example, other phase transfer catalysts may be used. Examples include tetrabutylammonium chloride, benzyl(trimethyl)ammonium chloride, tetrabutylphosphonium bromide, and tetrabutylammonium iodide. In addition, other hydroxide bases (e.g., KOH, or LiOH), bis(trimethylsilyl)amine bases (e.g., NaHMDS, KHMDS, or LiHMDS), tert-butoxide bases (e.g., Na, Li, or K tert-butoxide), carbonate bases (e.g., K₂CO₃, or Cs₂CO₃), may be employed. For aqueous NaOH, other concentrations ranging from about 15 wt % to about 50 wt % are also acceptable. Various solvents, including 2-methyltetrahydrofuran, or MTBE, may be employed, and temperatures may range from about 20 to about 70° C.

The present disclosure is not to be limited in scope by the specific embodiments disclosed in the examples, which are intended to be illustrations of a few embodiments of the disclosure, nor is the disclosure to be limited by any embodiments that are functionally equivalent within the scope of this disclosure. Indeed, various modifications of the disclosure in addition to those shown and described herein will become apparent to those skilled in the art and are intended to fall within the scope of the appended claims. To this end, it should be noted that one or more hydrogen atoms or methyl groups can be omitted from the drawn structures consistent with accepted shorthand notation of such organic compounds, and that one skilled in the art of organic chemistry would readily appreciate their presence.

What is claimed is:

1. (canceled)

2. (canceled)

3. (canceled)

4. (canceled)

5. A process for preparing a compound of Formula (I) or a salt thereof: 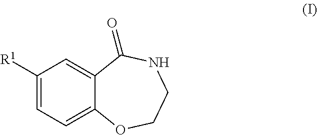
comprising:
a) deprotecting a compound of Formula (III) or a salt thereof: 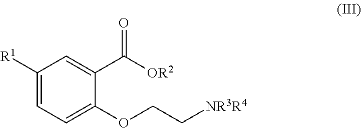
under reaction conditions sufficient to provide a compound of Formula (I) or a salt thereof, and 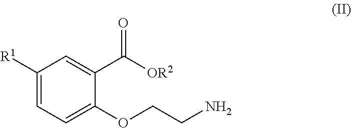
b) cyclizing a compound of formula (II) or a salt thereof, under reaction conditions sufficient to provide the compound of Formula (I) or a salt thereof, wherein:
R¹ is hydrogen or halo;
R² is hydrogen or alkyl;
R³ is a nitrogen protecting group; and
R⁴ is hydrogen, or R³ and R⁴ together with the nitrogen to which they are attached form N-diphenylmethyleneamine or a succinimide.

6. (canceled)

7. The process of any one of claim 5, wherein R³ is hydrogen, acyl, allyl, —C(O)O-alkyl, or benzyl; and R⁴ is hydrogen.

8. The process of claim 5, wherein R³ and R⁴ together with the nitrogen to which they are attached form a succinimide.

9-48. (canceled)
